(19) United States
(12) Patent Application Publication
(10) Pub. No.: US 2022/0384053 A1
(43) Pub. Date: Dec. 1, 2022

(54) TRAINING DATA COLLECTION REQUEST DEVICE AND TRAINING DATA COLLECTION METHOD

(71) Applicant: OLYMPUS CORPORATION, Tokyo (JP)

(72) Inventors: Osamu NONAKA, Sagamihara-shi (JP); Tomoko GOCHO, Tokyo (JP); Manabu ICHIKAWA, Tokyo (JP); Hirotatsu FUJIHARA, Ina-shi (JP); Ryo SAKURAI, Saitama-shi (JP)

(73) Assignee: OLYMPUS CORPORATION, Tokyo (JP)

(21) Appl. No.: 17/884,971

(22) Filed: Aug. 10, 2022

Related U.S. Application Data
(63) Continuation of application No. PCT/JP2020/010949, filed on Mar. 12, 2020.

(51) Int. Cl. G16H 50/70; G06N 5/02

(52) U.S. Cl. CPC G16H 50/70; G06N 5/022

(57) ABSTRACT

A training data collection request device, having a communication circuit receives information relating to symptoms of a specified patient, and a processor specifies a device that is capable of acquiring previous time series data of the patient, wherein the processor acquires, for another person who is using the similar type of device to the device that was specified, data and consultation information that has been collected using the similar type of device to create the training data.

### CROSS-REFERENCE TO RELATED APPLICATIONS

This application is a Continuation Application of PCT Application No. PCT/JP2020/010949, filed on Mar. 12, 2020, the entire content of all of which is incorporated herein by reference.

### BACKGROUND OF THE INVENTION

### 1. Field of the Invention

The present invention relates to a training data collection request device and a training data collection method, that, when a new event has been found during daily examination and consultation by a specialist such as a physician, can easily collect information representing a process that has led to this event, and can create training data for generating an inference model based on this information that has been collected.

### 2. Description of the Related Art

Technology is known for classifying information such as test and images into any of a plurality of categories using artificial intelligence related technology such as natural language processing and deep learning technology. However, activities where training data is learned in artificial intelligence, classification results of artificial intelligence are evaluated, and where classification is performed by switching artificial intelligence that has learned different training data, are troublesome activities for service providers, and place a significant burden on service providers.

A classification support device has therefore been proposed in order to reduce the load on service providers, whereby correspondence relationships, between characteristics of learning models that classify target data based on learning results that used target data and training data, are stored, target data is acquired from clients, and target data is classified by designating a learning model having characteristics corresponding to this target data (refer to Japanese patent laid-open No. 2018-028795 (hereafter referred to as “patent publication 1”)).

The classification support device described above can provide support when classifying into classification models that are already prepared. However, there is no description in this patent specification of, when a new event has occurred, easily collecting information indicating a process leading to this event, and creating training data for generating an inference model based on this information that has been collected.

### SUMMARY OF THE INVENTION

The present invention provides a training data collection request device and a training data collection method, that, when a new event has occurred, can easily collect information representing a process that has led to this event, and can create training data for generating an inference model based on this information that has been collected.

A training data collection request device of a first aspect of the present invention comprises a communication circuit receives information relating to symptoms of a specified patient; and a processor specifies a device that is capable of acquiring previous time series data of the patient, wherein the processor acquires, for another person who is using a similar type of device to the device that was specified, data and consultation information that has been collected using the similar type of device to create the training data.

A training data collection device of a second aspect of the present invention comprises a communication circuit receives information relating to symptoms of a specified patient based on results of having performed diagnosis on the patient; and a processor specifies a device that is capable of acquiring previous time series data of the patient, wherein the processor acquires, for another person who is using a similar type of device to the device that was specified, data and consultation information that has been collected using the similar type of device to create the training data.

A training data collection method of a third aspect of the present invention comprises receiving information relating to symptoms of a specified patient; specifying a device that is capable of acquiring previous time series data of the patient; and requesting collection of time series data of another person having a similar type of device to the device that has been specified to create the training data.

A non-transitory computer-readable medium of a fourth aspect of the present inventions stores a processor executable code, which when executed by at least one processor which is provided in a training data collection device, performs a method, the method comprising receiving information relating to symptoms of a specified patient; specifying a device that is capable of acquiring previous time series data of the patient; and requesting collection of time series data of another person having a similar type of device to the device that has been specified to create the training data.

A non-transitory computer-readable medium of a fifth aspect of the present invention stores a processor executable code, which when executed by at least one processor which is provided in training data collection device, performs a method, the method comprising receiving information relating to symptoms of a specified patient based on results of having performed diagnosis on the patient; specifying a device that is capable of acquiring previous time series data of the patient; and acquiring, for another person who is using a similar type of device to the device that is specified, data and consultation information that has been collected using the similar type of device to create the training data.

### BRIEF DESCRIPTION OF THE DRAWINGS

FIG. 1A and FIG. 1B are block diagrams showing overall structure of a training data collection system of one embodiment of the present invention.

FIG. 2A and FIG. 2B are drawings showing examples of data for learning, in a training data collection system of one embodiment of the present invention.

FIG. 3 is a drawing showing one example of data that is stored in a DB section of the training data collection system of one embodiment of the present invention.

FIG. 4A and FIG. 4B are drawings showing display examples on a display section for physician operations in the training data collection system of one embodiment of the present invention.

FIG. 5A and FIG. 5B are drawings showing chronological change on a display section for physician operations in the training data collection system of one embodiment of the present invention.

FIG. 6A and FIG. 6B are drawings showing display examples of menu screens on a display section for physician operations in the training data collection system of one embodiment of the present invention.

FIG. 7A and FIG. 7B are drawings showing display examples of patient section screens on a display section for physician operations in the training data collection system of one embodiment of the present invention.

FIG. 8 is a drawing showing a display example of a diagnosis input screen on a display section for physician operations in the training data collection system of one embodiment of the present invention.

FIG. 9A and FIG. 9B are flowcharts showing one example of operation of a control section, in the training data collection system of one embodiment of the present invention.

FIG. 10A and FIG. 10B are flowcharts showing another example of operation of a control section, in the training data collection system of one embodiment of the present invention.

### DETAILED DESCRIPTION OF THE PREFERRED EMBODIMENTS

In the following, an example where the present invention has been applied to a data collection system that utilizes an IT platform, or the like, will be described as one embodiment of the present invention. The data collection system of this embodiment collects output data of many devices, and performs inference using an inference model that has been generated by performing learning with this data that has been collected as training data. There are cases where a specialist such as a physician discovers mental and physical changes from a normal state, such as symptoms the subject (user, patient) is not aware of at the time of daily consultation and medical examination. In this case, if the physician, or the like, inputs an examination device that the subject is using to a data collection system, it is possible to acquire previous time series data that this subject has learned using the examination device. By looking at this data it is possible to confirm processes performed by the subject up to that point, which will become useful when determining illness etc.

Also, with the data collection system it is also possible for another person to collect examination data that has been examined using a similar device, without being limited to the subject, and display this data that has been collected. A specialist can perform more highly accurate diagnosis by referencing this data of another person.

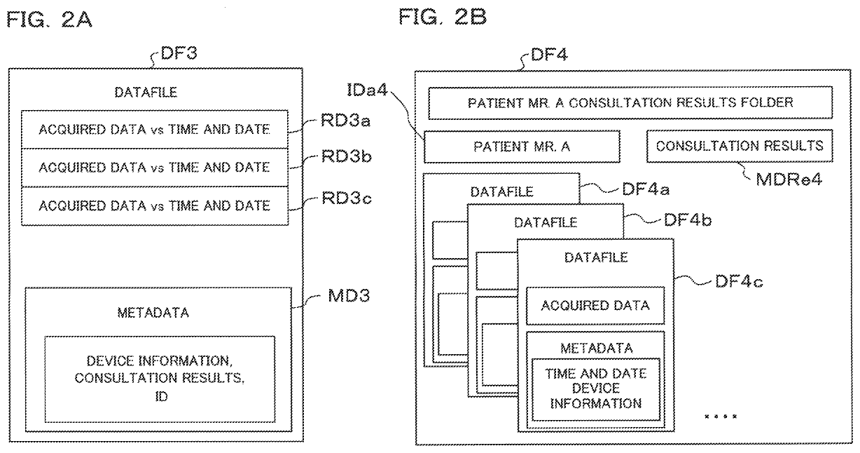

Also, a specialist such as a doctor can request generation of an inference model, with data that has been collected by the data collection system as training data. Also, in the case of unfamiliar symptoms, it is possible for this inference model to infer what type of illness there is, transitions in symptoms in the future (for example, when symptoms will worsen, and when to go to hospital etc.) and treatment methods etc. It should be noted that it goes without saying that an application is possible where people that are not specialists are assisted, based on assistants to specialists following specialist guidance. Also, assistants and people other than specialists copy the methods of specialists, and there are methods for checking the act of copying by inferring these actions, and in various cases such as this it is only necessary for the history of those actions to be clarified. It is made possible to store classifications and IDs etc. of people having attached annotation to metadata of data files shown in FIG. 2A, and schemes such as applying weighting for training data on annotation results that were performed by a specialist may also be performed.

Also, with this embodiment, in the data collection system accurate health status is ascertained by taking into consideration the condition of the user (patient), and in order to provide customized information, for example, examination data relating to health state is monitored on a daily basis using examination devices such as a first device and a second device, and resulting data is collected. Information relating to health is provided by the data that has been collected by this data collection system. That is, this training data collection system monitors examination data relating to health status of the user, on a daily basis, using a plurality of devices.

It is possible to display advice relating to health of the user using history data that has been acquired as a result of this monitoring. Also, when presenting advice, data that has been collected as a result of monitoring is input to an inference engine in which an inference model has been set, and it is possible to display advice based on inference results from the inference engine.

It should be noted that this data that has been collected may be numerical values for some specified items, and metadata is associated with this acquired data. Determination may also be performed including this metadata. Although this metadata and acquired data are collectively referred to as acquired data, in actual fact these data groups may be handled in accordance with files and data formats, and data groups may be handled by collecting them into folders. As metadata, there are information as to which individual the data belongs to, acquisition time and date information, type of device that outputs data, and the type of the data that is output, etc. Metadata may also include data on measurement environment etc. Naturally, as a system, in a case where it is possible to restrict change elements that these metadata represent, there will be data that can be omitted.

Incidentally, as examination devices that the user utilizes, there may be cases where the devices are installed in the home or in the workplace (including schools etc. that are being attended). As examination devices that are installed in the home etc., there are electronic sphygmomanometers, electronic clinical thermometers, stool and urine examination devices installed in toilets, etc. As well as in the home etc. various examination devices are used in periodic medical examinations, health screening, and diagnosis at the time of blood donation etc. Further, various examination devices are also used when the user has been admitted to a medical institution. In this way, use of various examination devices is commonplace, and it is common for the user to normally use examination devices every day.

Also, in a case where range of user behavior and content of behavior etc. is accompanied with outbreak of a specified illness and risk of deterioration, GPS and charge settlement functions provided in a mobile terminal possessed by the user can also constitute examination devices. Recently, wearable terminals tend to be provided with the above described functions. In a smart house or the like, health management cameras are provided in washstands, and it is possible to determine room temperature, electricity, gas, and water usage, and whether or not, and when, to take a bath etc. It is also possible to use surveillance cameras and vehicle mounted cameras etc. as examination devices. While it is possible for miscellaneous devices to perform life-monitoring in this way, it is not realistic due the workload, energy and memory capacity involved in sensing and storing if data of all devices is collected together, and the occurrence of problems with communication load. Also, the user is not aware of which information is important. Conversely, if the user is aware of which information is important, there is a danger of receiving a negative reaction. Also, there are problems of individual information, and generally speaking ii it not desirable to use data from these devices willfully, and it is preferable to utilize information that has been prescribed under conditions regulated in agreements with business organizations that perform services, systems, and specified users who constitute subjects.

In this embodiment, a data collection system collects information from a plurality of types of examination device that the user uses, and stores this information in a database. As was described previously, it is then possible for a specialist such as a physician to retrieve data as required, and to give various advice to the user by performing inference using this retrieved data. Generation of an inference model for giving this advice can be requested to the learning device by way of the data collection system. It is also possible for an individual or organization other than a specialist to acquire information and request generation of an inference model in order to select behavior that will reduce risk.

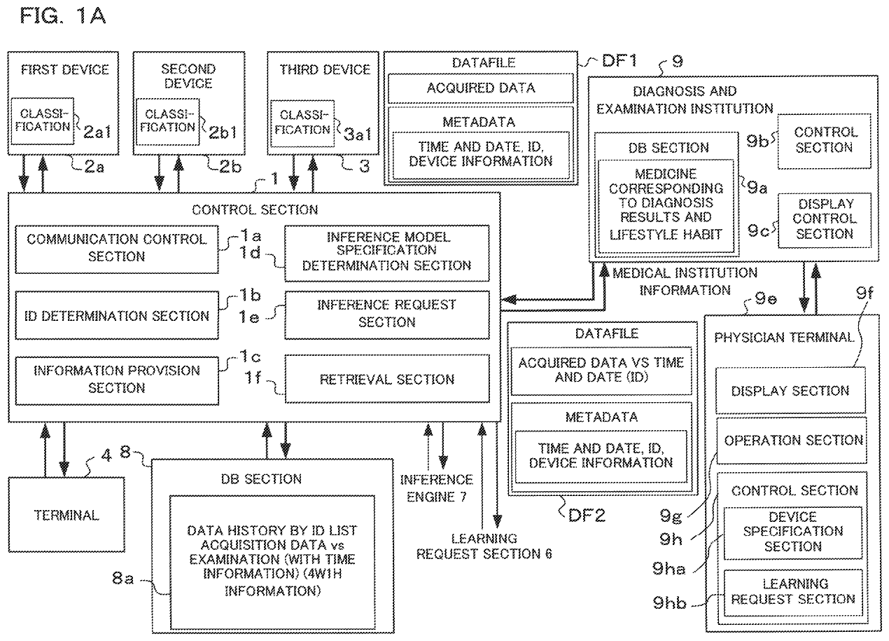

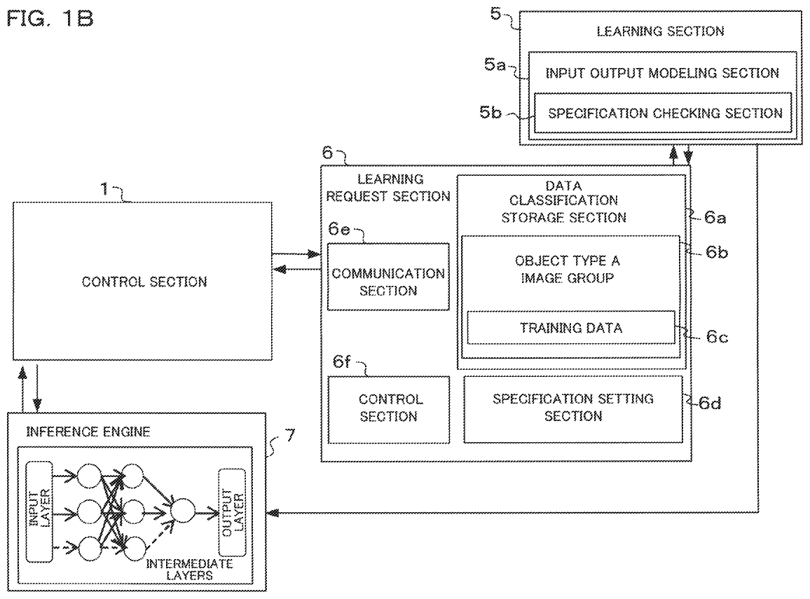

Next, the structure of a training data collection system of one embodiment of the present invention will be described using FIG. 1A and FIG. 1B. This training data collection system comprises a control section 1, first device 2a, second device 2b, third device 3, terminal 4, learning section 5, learning request section 6, inference engine 7, database (DB) section 8, diagnosis and examination institution (including medical institution etc.) 9, and physician terminal 9e. It should be noted that the control section 1 depicted in FIG. 1B is the same as the control section 1 depicted in FIG. 1A, but the detailed structure of internal sections has been omitted from FIG. 1B.

Among each of the sections of the data collection system, the control section 1 is arranged within a server. The first device 2a, second device 2b, third device 3, terminal 4, learning section 5, learning request section 6, inference engine 7, DB section 8 (this can also be expressed as a recording section or storage section), and diagnosis and examination institution 9 are capable of connecting to a server by means of a network such as the Internet. However, this embodiment is not limited to this structure, and, for example, one or several among the control section 1, first device 2a, second device 2b, third device 3, learning section 5, learning request section 6, inference engine 7, and DB section 8 may be arranged within a server, and alternatively, may also be arranged in electronic devices such as a separate server or personal computer. Further, the diagnosis and examination institution 9 may have a server function.

Also, the first device 2a, second device 2b, third device 3, terminal 4, and diagnosis and examination institution 9 may have the same functions as the control section 1, may have the same storage functions as the DB section 8, and may execute the control that will be described below as performed by the control section 1. For example, control which will be described below may be executed as being performed by the control section 1, which is on the cloud, in cooperation with the first device 2a, second device 2b, third device 3, terminal 4, and diagnosis and examination institution 9, etc., as edge devices (terminals). This control is often optimized for each system, because of limitations such as communication speed at the time of cooperation, hardware structure of each edge device, power consumption etc. However, here, in order to be able to simplify the description the control section 1 is described as being dedicated to performing the control below.

The control section 1 is a controller (processor) that controls the data collection system of this embodiment, and it is assumed to be an IT device comprised of a CPU (Central Processor Unit) that provides files and data etc. to other terminals by means of a server etc. or network, memory, an HDD (Hard Disc Drive) etc. However, the control section 1 is not limited to this structure, and in the case of constructing a small-scale system it is possible to be configured with something like a personal computer. The control section 1 has various interface circuits, and can cooperate with other devices, and various arithmetic control is possible using programs.

The control section 1 receives information from each cooperating device, organizes the information, creates necessary information, and provides this information to the user. The control section 1 outputs requests to each cooperating device, and also has functions such as operating each device. In this embodiment, it is intended to have a high degree of freedom and usability with respect to the system, and it is possible to connect between devices such as the first device 2a, the terminal 4 that the user has, and the control section 1 using wireless communication or wired communication. A wireless LAN or mobile telephone network is intended as communication for this purpose, and it is also possible to use short-distance wireless such as Bluetooth (registered trade mark) or infra-red communication in accordance with conditions. The communication circuits have been omitted in FIG. 1 as they would make the description of the communication sections, constituted by antennas and connection terminals etc., complicated, but communication sections that have communication circuits are provided at arrow sections showing communication in the drawings.

The control section 1 has a communication control section 1a, an ID determination section 1b, an information provision section 1c, inference model specification determination section 1d, inference request section 1e, and retrieval section 1f. Each of these sections may be implemented with software, using a processor having a CPU etc. within the control section 1 and programs etc., or may be implemented as hardware circuits, or may be implemented by collaboration between software and hardware circuits. Also, as was described above, the control section 1 is configured with a processor having a CPU etc., and implements functions possessed by the communication control section 1a, ID determination section 1b, information provision section 1c, inference model specification determination section 1d, inference request section 1e, and retrieval section if (for example, input section (input section at the time of cloud control), device specification section, learning request section, display control section etc.). Also, the processor is not limited to being a single processor and control may be divided over a plurality of processors, and functions of each section may be implemented by respective collaborative operation.

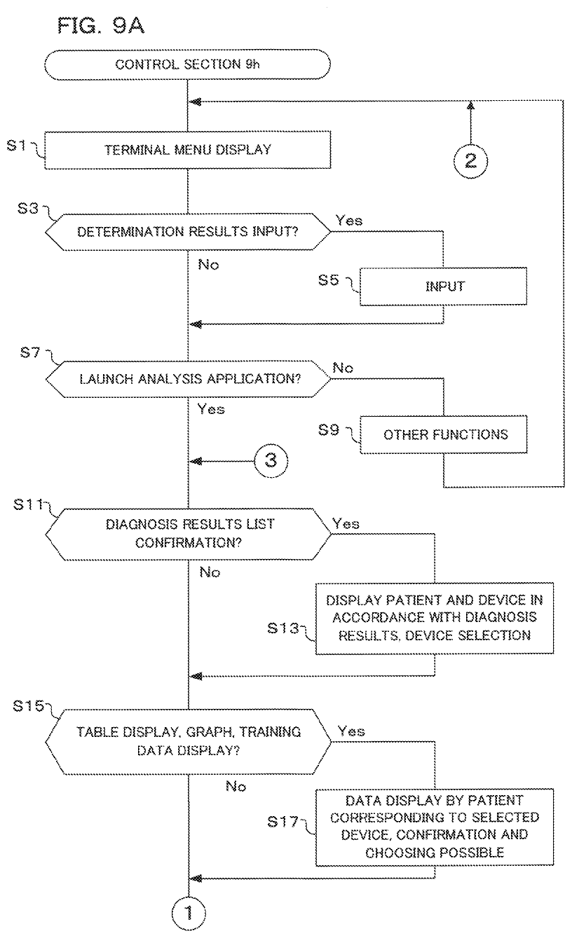

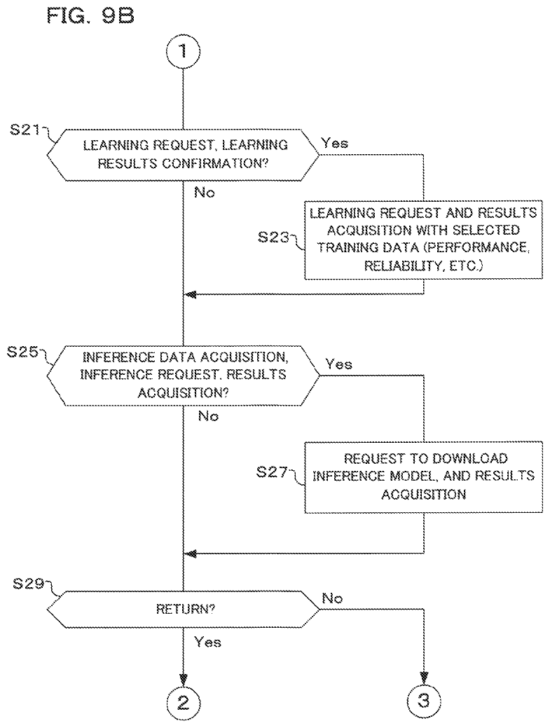

For example, FIG. 9A and FIG. 9B are flowcharts showing one example of operation of the control section, in the data collection system of one embodiment of the present invention, but these flowcharts are described simply such that a single control section (for example, the control section 1 in FIG. 1) executes all of the respective steps. However, in actual fact, in each step there is collaboration with other blocks (for example, the first device 2a˜third device 3, diagnosis and examination institution 9, DB section 8, terminal 4, etc.). Also, since each block itself has similar functions to the control section 1, each step in the flowcharts may be distributed across each block. This involves determining what should be executed, in accordance with conditions and background environment of the system, so that arguments such as which blocks should perform which steps are performed in a general manner, on the cloud or edge device (terminal).

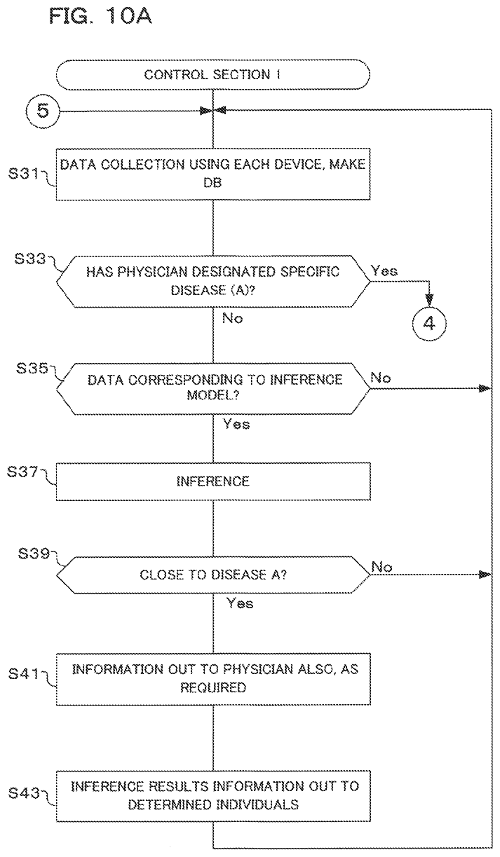

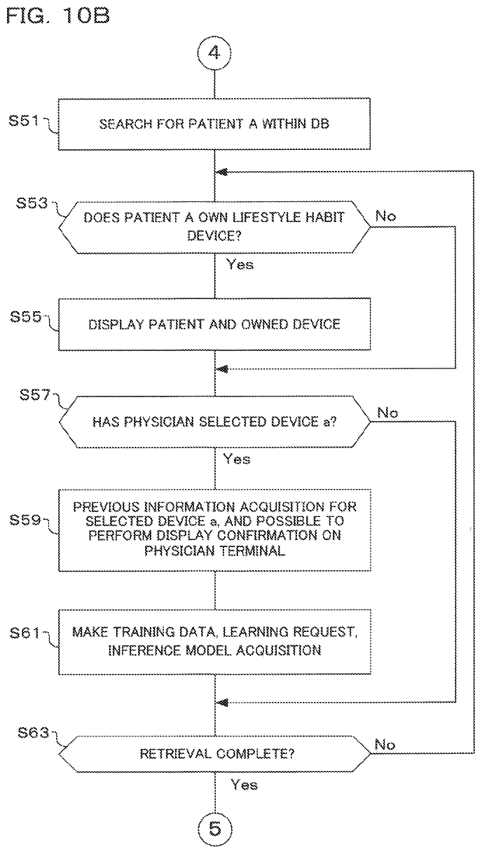

It should be noted that in FIG. 1A, for each section within the control section 1, direction of signals for fulfilling respective collaborative functions have been omitted, but this will be described separately with the flowcharts. For example, in steps such as S31 in FIG. 10A, the ID determination section 1b collects information for every identical user from the first device 2a, second device 2b etc.

The communication control section 1a has a communication circuit etc., and transmits and receives data etc. with communication sections (communication circuits) provided within the first device 2a, second device 2b, third device 3, terminal 4, learning section 5, learning request section 6, inference engine 7, database (DB) section 8, and diagnosis and examination institution 9. The communication control section 1a provides a function as a data input section (input circuit) for inputting output data from devices. Also, the communication control section 1a functions as an information acquisition section for acquiring information. It should be noted that although each of the devices and sections, such as the first and second devices 2a and 2b, the third device 3, terminal 4, and diagnosis and examination institution 9, also have respective communication sections, they have been omitted form FIG. 1 since it would complicate the drawing.

The ID determination section 1b collects information each time for the same user, from the first device 2a etc. In order to specify individuals that have acquired information using the first device 2a, second device 2b, third device 3, and diagnosis and examination institution 9, an ID is assigned to each individual. In this embodiment, since individual user data is handled, management of which user's information is received and which user guidance is output to is performed by the ID determination section 1b. This determination of a specified user is performed by the first device 2a, second device 2b, and third device 3 having a biometric authentication function, the user inputting an ID using the terminal 4, the user transmitting an ID by means of a communication section with the first or second device 2a or 2b, or the terminal 4 reading in a unique code. It should be noted that in order to protect individual information management is made stricter by encrypting necessary section, but these processes involve generic technology and so detailed description is omitted.

As an ID for each device, as will be described later, each device stores type information, and information relating to device name of that device, and unique information etc. that represents what individual is using that device, may be determined using the type information. It may be made possible to understand functions and performance etc. of incorporated sensors from model names, and to understand installation location and usage environments etc. from individual information, and these items of information may be retrievable by means of a network or the like. If model names are understood, it is possible to determine information of various devices, it is possible to determine latitude and longitude, whether indoors or outdoors, the season, climate, temperature characteristics etc. from installation location and usage environment, and correction of output information of these devices may be performed by adding the results of these determinations.

The information provision section 1c has a function to acquire information of the user (results acquired by other devices may also be referenced) in order to provide correct information to the user. Also, the information provision section 1c acquires examination data of the user (specified using ID) that has been acquired from the first device 2a etc. or diagnosis and examination institution 9, and diagnosis results from physicians etc. The information provision section 1c functions as an input section (input interface, input section at the time of cloud control) for inputting information relating to symptoms of a specified patient based on results of a physician having performed diagnosis on the patient (refer to S31 in FIG. 9B, for example).

Also, if there is a request from the diagnosis and examination institution 9, the information provision section 1c provides examination data that has been acquired by the first device 2a etc. and data etc. stored in the DB section 8, to the diagnosis and examination institution 9. The information provision section 1c also provides similar data if there is a request from a physician etc. using the physician terminal 9e by means of the diagnosis and examination institution 9.

Further, the information provision section 1c determines health status of the user using examination data that has been acquired, various information that has been acquired from the diagnosis and examination institution 9, and information relating to owned equipment stored in the DB section 8 and profile information etc. of the user. Health status includes current illness, and illness that may possibly develop, and once health status has been determined information relating to the health status is provided to the user. Also, in the event that an illness etc. of the user has been determined, information relating to examination and facilities where treatment should be received is provided to the user, as required.

Also, in order to confirm health status of a user in a specified condition, if it is made possible for the control section 1 to inquire to the diagnosis and examination institution 9 about information such as current attendance conditions and prescriptions etc., previous health diagnosis results etc., determination of association with device data becomes easy. It is therefore possible to take measures to deal with security problems resulting from allowing the user operating the terminal 4 to have this cooperation, and a physician who is operating (IT devices of) the diagnosis and examination institution 9 performing operations such as consenting to cooperation.

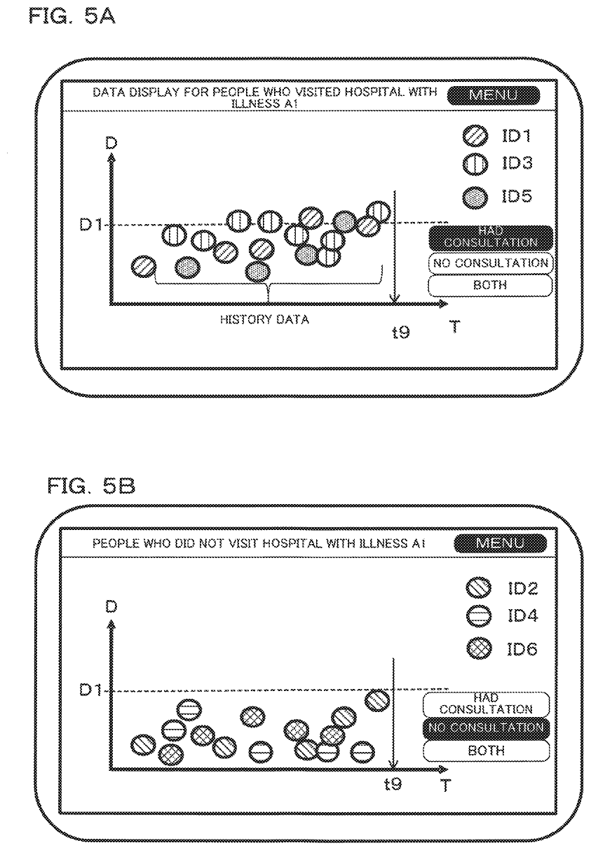

Specifically, the information provision section 1c may provide information relating to health, namely, information such as when it is arranged to visit a facility in order to receive examination or treatment, or information for recommending a facility that is suitable for undergoing examination or treatment, to the user. The information provision section 1c acquires examination data that has been transmitted from the first device 2a etc. or the diagnosis and examination institution 9. As will be described later, this data is examination data that has time information attached (time series information), and is in a data structure so that it is possible to form into graph, such as shown in FIG. 5A and FIG. 5B. It should be noted that with this embodiment, although it is assumed that the control section 1 performs provision of information to the user using information from devices such as the first device 2a etc. and the diagnosis and examination institution 9, there may also be modified examples, such as a server owned by the diagnosis and examination institution 9 similarly collecting information.

Also, in order to provide these items of information, the information provision section 1c collects examination data from the first device 2a and second device 2b etc., and stores this data in the DB section 8. Depending on the first device 2a and the second device 2b the frequency of information acquisition and the number of data may differ. That is, increase and decrease in specified health associated numerical values obtained by various devices is managed in time series, and it becomes possible to organize numerical values, measured by changing devices, for each device.

In a case where lifestyle habits such as behavior patterns, eating habits, bedtimes, and times of meals etc. for addresses and places of work of users, are stored in the DB section 8, the information provision section may acquire these items of information from the DB section 8, and these items of information may be acquired on the Internet. The information provision section 1c may generate information such as for facilities etc. provided to the user by adding this information that has been acquired. Acquisition of these items of information can be completed with generic or widely known technology. It may also be possible for customization of information such as for a facility etc. that has been generated by acquisition of these items of information to be performed by the information provision section 1c. Profile information relating to this facility is acquired as medical institution information from the diagnosis and examination institution 9.

The information provision section 1c acquires examination data constituting time series patterns for a specified period of the user. This time series pattern to be acquired is not simply data that has been acquired by measurement a single time, but is formed using respective examination data that has been acquired at a plurality of different times, and is utilized to the extent of change in the examination data pattern. By using time series patterns made up of a plurality of examination data it is made less likely to be affected by errors arising due to change in measurement environment and conditions. Further, health status is inferred for a time in the future (on extension of specified period into the future) from an end time of the specified period, making it possible to predict health status for the future.

Also, training data can be created if information on time when the user is admitted to an examination and medical institution is attached to a time series pattern that has been acquired, as annotation information training data. If there is an inference section having an inference model that has been generated by learning using this training data, it is possible to infer what will occur at a time after (on extension of specified period) a specified period (period for acquiring a time series change pattern). Also, if the name of a user's illness is known, it is possible to generate training data that has this information attached as annotation information. It is possible to generate an inference model for inferring health information of an illness etc. by learning using this training data. It should be noted that when generating an inference model used here, specification for specified input and output information is regulated, and learning is performed.

In this way, when performing machine learning and deep learning, information having annotation attached to data is made training data. When groups of data such as time series data have been collected, it is considered that respective data contains some information that contributes to that annotation result, and data containing that information constitutes training data. However, there are cases where errors or noise are superimposed on the data because of some kind of problem, and that data may not be suitable for use because of faults etc. at the time of information detection or transmission. Training data may therefore be selected from among data that satisfies conditions such as data format that has been set in advance as required, data specification, data type, and data size range etc.

Accordingly, with this embodiment, time series change patterns for examination data of the user is input to an inference section, the inference section performs inference, and a transmission information determination section is provided to determine transmission information for a time after a specified period, based on the results of the inference. As a result, it is possible to provide a system, device, method, and program etc. that can transmit prediction information for a time after examination acquisition of a time series pattern.

The information provision section 1c of this embodiment inputs a change pattern for examination data to an inference engine 7 in which an inference model that was generated by the learning section 5 is set, and obtains inference results relating to advice and provides this advice to a user corresponding to the examination data that has been input. There are cases where this service uses individual information, and there are cases where agreements for personal information usage are required in order to receive provision of advice etc. In that sense profile information of a user is sometimes important. Also, in a case where the user is a child or elderly, advice may be passed on to a person or care-giver looking after that user. Effective information such as advice is also passed on in accordance with information that has been managed with profile information of the user.

The inference model specification determination section 1d determines specifications of an inference model that is generated, when the inference request section 1e requests generation of an inference model to the learning section 5 by means of the learning request section 6. The control section 1 acquires bio-information of the user from the first device 2a etc., and stores this bio-information. The control section 1 requests generation of various inference models to the learning section 5, by means of the learning request section 6, with the bio-information that has been stored as training data. Also, as will be described later, there are cases where a physician or the like requests generation of an inference model from the physician terminal 9e (refer, for example, to S21 and S23 in FIG. 9B). In this case, the inference model specification determination section 1d may determine specifications of the inference model. This inference model is learned using training data, and expected results of this inference model are that specified specimen information and bio-information are input, and output is made diagnosis assisting information.

If there is a useful inference model for a patient of a similar case, and incipient patients, health related information such as for many people themselves or their supporters is input to the inference model, and there is a line of thought that it is possible to improve health by using these inference results. This concept came about with the background that it has become possible to acquire health related information of many people from various monitoring devices (for example, the first to third devices in FIG. 1A) throughout the world, and useful information has become easily accessible by many people using information terminals, as a result of connecting together various devices on the Internet stemming from the creation of the Internet of Things (IoT) using advancements in IT technology.

With this concept, it becomes possible to boost the health consciousness of each individual, and in order to confirm the need for a hospital visits it is possible to use monitoring devices (for example, the first to third devices in FIG. 1A) as tools, and it is possible to prevent people unnecessarily going out to clinics etc. where contagion is more likely. For example, assuming the first to third devices (FIG. 1A) to be wristwatch type terminals, if they can be used as sleep and heart rate monitors then with recent studies they can be made into devices that give notification so as to warn of the possibility of influenza using this monitor data, and so it becomes possible to deal with influenza. Specifically, if cases where a physician determines influenza on the basis of actual device data, and cases where they determine influenza without actual device data, can be distinguished (if this type of approach is assumed, it may be made possible to input consultation results and determination results such as a physician doubting influenza but it actually being influenza), it is possible to prevent cases where there is a risk of contact with other patients at medical institutions even if possibility of influenza is low, and to prevent cases where people go out without wearing a mask, posing a risk to other patients, even though there is a possibility of influenza. Also, a physician may also reference the above mentioned health-related information (information that has been obtained every day in time series) at the time of consultation. Further, in addition to inquiries etc. (face to face meetings or video calls), diagnosis may be performed using influenza examination kits (infection determination kits) etc.

Because there are valuable diagnosis results that have been obtained in this way, similar consultation performed through similar processes, or resulting knowledge through similar processes, are used extensively, and this is a major feature of this embodiment. Specifically, for various medical institutions and physicians, results of having performed consultation and diagnosis for various patients are made into training data, and if other physicians refer to big data, which is a collection of these training data, and inference models that have been created by means of the above described processes, it is also possible to deal with problems of the lack of physicians in recent years, and the need to improve the health consciousness of people.

When generating an inference model, the inference model specification determination section 1d determines what type of specification inference model has been requested. For example, in a case where time series bio-information is stored, the inference model specification determination section 1d determines specification of an inference model in order to infer what type of examination data (values) will result, and how many days later the user will receive treatment at a medical facility. Also, the inference model specification determination section 1d determines specifications for generating an inference model for inferring, based on time series bio-information, what illness a patient currently has, and what the possibilities are of the patient having what illnesses (when) in the future, and recommended facilities in order to receive necessary examination and treatment in the event that an illness might occur again.

The inference request section 1e requests generation of an inference model, of the specifications that have been determined by the inference model specification determination section 1d, to the learning section 5 by means of the learning request section 6. Specifically, the inference request section 1e requests generation of an inference model to the learning section 5 by means of the learning request section 6, in the event that a specified number of items of bio-information that have been acquired by the first device 2a are stored, and receives an inference model that has been generated by means of the learning request section 6. This inference model that has been received is transmitted to the inference engine 7. It should be noted that the control section 1 prepares a plurality of inference models, and may select an appropriate inference model in accordance with information that should be provided to the user. Also, if the control section 1 communicates with the learning section 5 directly, the inference model may be received directly from the learning section 5. Further, as will be described later, there are cases where a physician or the like requests generation of an inference model from the physician terminal 9e (refer, for example, to S23 in FIG. 9B). In this case, the inference request section 1e may request generation of an inference model to the learning section 5 by means of the learning request section 6.

The inference request section 1e functions as an inference request section to make data, that is what was expected to have been collected, into training data, and perform learning requests of an inference model corresponding to a training data collection system. The inference request section 1e functions as an inference model acquisition section that acquires an inference model generated by learning using training data that has been collected. The inference model acquisition section learns time series transition patterns for values of training data that have been collected and acquires an inference model. The inference request section 1e functions as a learning request section that makes time series data of another person having a similar device to a device that has been specified, and consultation information, into training data, and requests learning (refer, for example, to S61 in FIG. 10B).

When an illness currently being suffered from, and what illnesses there is a possibility of suffering from (when) in the future, and further, whether examination and treatment are required, have been identified, based on bio-information of a user that has been acquired by the first device 2a, second device 2b, and third device, the retrieval section 1f performs retrieval of examination institutions and medical institutions having the facilities required for examination and treatment, from within the database stored in the DB section 8. These items of information may be obtained by inference using the inference engine 7, but there are also cases where this information is coincident with data that is stored. Since there is also this type of case, with this embodiment it is made possible to search using the retrieval section 1f.

Also, as will be described later, there are cases where a physician or the like, at the physician terminal 9e, searches for examination data that was acquired using an examination device being used by the patient (refer to S17 in FIG. 9A, for example). In this case, if there is a search request via the diagnosis and examination institution 9 the retrieval section 1f performs search in accordance with the request. The retrieval section 1f functions as a device specification section that specifies devices that are capable of acquiring previous time series data of a patient (refer, for example, to S51, S53 and S55 in FIG. 10B). The device specification section displays devices for data collection from a list of devices on the display section, and specifies devices from among these devices that have been displayed (refer, for example, to S55 on FIG. 10B).

The device specification section specifying devices that can acquire previous time series data of a patient is effective in searching for data of other people who have been examined using the “same device”, in cases where a lot of people use a shared device, and in making data into big data and reducing noise data, as a result of searching for data of other people who have been examined using a “device of the same model number” or “device of the same specifications”, and is also effective for increasing the amount of training data etc. Also, in a case where it is known that specified conditions will have an effect on illness and health status, data may be appropriately chosen in order to make up for those conditions. For example, in a case where it would be more effective to sort data in accordance with search conditions such as specified gender, specified age, specified region etc., those conditions are provided at the time of search.

The first device 2a and second device 2b are devices for acquiring a user's health related information, for example, examination data such as vital information and specimen information. The first device 2a and second device 2b are examination devices of a specified specification, and are devices that are capable of examination of the same kind of (similar) health related information. The first device 2a stores a classification 2a1, and the second device 2b stores a classification 2b1. Classification 2a1 and classification 2b1 are information relating to type, model number, and examination items of a device, and when examination data of the user is transmitted to the control section 1 using each device, this classification is also transmitted.

When groups of examination data that have been acquired by the first device 2a and second device 2b are for respectively different examination times, examination such that both data can be interpolated is preferable. Also, the first device 2a and second device 2b should examine identical examination items, and even if heart rate has been measured while measuring blood pressure, for example, both data can be respectively interpolated. It should be noted that in FIG. 1, as a device for acquiring examination data of the user only the first device 2a and second device 2b have been depicted, but the embodiment is not limited to two devices and there may be three or more devices. Also, as will be described later, as a device for acquiring examination data of someone other than a user, in this embodiment the third device 3 has been assumed.

It should be noted that there are cases where it is possible to make confirmation of health status more accurate, by acquiring similar data continuously. For example, numerical values representing health status will change for various reasons, such as with the four seasons of the year, with the three meals, breakfast, lunch, and dinner, in a day, before or after eating, or while working or when not working, for a work day and when teleworking, during holidays, etc., and so it is possible that abnormalities that people are unaware of with a normal examination will be found as a result of acquiring data continuously. Considering these types of conditions, it is desirable to continuously acquire similar data under various conditions. However, devices and equipment that acquire data may differ depending on conditions, and differences and errors may arise due to environmental changes for each condition, and various restraints, and comparison may not be possible on the same basis.

Therefore, together with being able to acquire a time series first examination data group of a subject using a first device, it is made possible to acquire a time series second examination data group of the subject using a second device that is capable of examination such that it is possible to interpolate the first examination data group, and by using these data groups it is possible to have a relationship where the first examination data group and the second examination data group respectively make up for any shortfall in examination times or examination items. Depending on conditions, a means of determining change in similar numerical values that are different for a first device and a second device becomes necessary, but by correcting first and second respective examination data groups errors are resolved and it is possible to expand and replenish information. Also, examination data that has been corrected is input. and reliability at the time of inference is calculated, and transmission information may be determined in accordance with this reliability. If reliability is low, it can be considered to be because correction has not been properly performed and inference results are not suitable for being provided. When correcting examination data, it becomes possible to handle cases such as where errors are simply accumulated or sensor gains are different etc. by performing basic arithmetic operations on respectively shared numerical values of data included in the examination data groups.

As health related information acquired by the first device 2a etc. there is various information, for example, vital information such as user body temperature, blood pressure, heart rate etc. Also, as health related information there is various specimen information such as the user's excreta, such as urine and feces, and phlegm and blood etc. In the case of feces, the first device 2a and the second device 2b acquire the color, shape, and amount of the feces, and time and date information. The first device 2a and second device 2b may acquire information in accordance with instruction from the control section 1, may acquire information in accordance with user operation, and may acquire information automatically. Further, the first device 2a etc. may collect and utilize personal life records (PLR), that have had various activity data of daily life, such as activities, diet, sports activities etc., that are part of daily life, or at an office/school, added to information “personal health records (PHR)”, which is medical and health information. The information that has been acquired is transmitted to the control section 1 by means of the communication section (omitted from the drawings) within the first devices 2a etc.

In a case where the first device 2a and second device 2b have obtained information relating to the user, the information provision section 1c of the control section 1 presents information relating to health to the user's information terminal 4. This presentation of information will be described assuming that user behavior is assisted, but various modifications can be considered. As information relating to health, there is information relating to medical facilities that will be recommended, and information relating to routine lifestyle habits.

The third device 3 may also be a device for acquiring data of a different person to the user who is using the first device 2a and second device 2b. There may be cases where the user using the first device 2a and the second device 2b commences new use of third device 3, or there may also be cases where that user uses the third device 3 temporarily. A single third device 3 is depicted in FIG. 1A, but there may also be a plurality of the third devices 3, and an unspecified large number of third devices 3 are represented in a unified manner in FIG. 1A.

It should be noted that the third device 3 also stores a classification 3a1. Classification 3a1 is information relating to type, model number, and examination items of the third device 3, and when the third device 3 transmits examination data of the user to the control section 1, classification information is also transmitted.

In a case where wearable terminals are used as the first device 2a, second device 2b, and third device 3, depending on the mounting regions of the wearable terminals they may be adhered to the skin or close to the body, and it becomes possible to obtain vital information such as body temperature, heart rate, blood pressure, brain waves, gaze, and respiration. Also, as weighing scales, sphygmomanometers, and measurement device that measure arterial stiffness, which means hardness of arterial walls, dedicated precision apparatus are provided in health facilities, public baths, pharmacists, and shopping malls etc., and there are also cases where specialist measuring staff are on site with the devices. In these types of facility, users are comfortable using measurement devices in their spare time etc., and it is often the case that users try to stay in good shape based on measurement results at this time. The first device 2a, second device 2b, and third device 3, may be these measurement devices.

Also, there are cases where the first device 2a, second device 2b, and third device 3 request the user to fill in a questionnaire before and after using the dedicated terminal or the like. In this type of case, it is possible to specify profile information and other information of the user based on what is written in the questionnaire. This type of information collection is not limited to the first device 2a and can also be performed by the control section 1. If it is possible to listen to information etc. as to when medical institutions and examination institutions etc. are performing examinations, it is also possible to use these items of information.

The first device 2a, second device 2b, and third device 3 may also be clinical thermometers and sphygmomanometers being used under the guidance of a physician, for users suffering from already specified diseases. Also, in cases such as the color of faces and fingernails, facial expressions, and images of affected parts, that have been photographed by a camera of a smartphone, and recording voice at the time the throat becomes abnormal using a microphone, a portable terminal (smartphone) constitutes the first device 2a, second device 2b, and third device 3.

There has recently been development in simplified health administration devices and health information acquisition devices, and there are cases where these devices are mounted in wearable devices, and many cases where these types of devices are also not treated as stand-alone devices but as peripheral devices of a smartphone, and so these may also be assumed to be portable terminals. There are also cases where simple measurement devices, not wearable devices, are installed where people congregate, to provide a health information service. The first device 2a, second device 2b, and third device 3, may be these types of devices.

Information such as user ID, device ID, and output data etc. from the first device 2a, second device 2b, and third device 3 is transmitted to the communication control section 1a of the control section 1. At the time of transmitting this information, the information is transmitted in the file format of a data file DF1. A data file DF1 is made up of acquired data RD1 and metadata MD1. The acquired data RD1 is data that has been acquired by each device, and the metadata MD1 includes time and data information for when the acquired data was acquired, ID specifying the person who underwent examination, device information for the device that acquired the acquired information, etc. Other formats for the data file DF will be described later using FIG. 2A and FIG. 2B.

The diagnosis and examination institution 9 has a DB section 9a, a control section 9b, and a display control section 9c, and includes facilities where the user undergoes medical examination, consultation, and examination, for example, an inspection facility or a medical care facility, and pharmacies. Physicians or the like engaged in medical care at the diagnosis and examination institution can exchange information with the diagnosis and examination institution 9 using a physician terminal 9e, which will be described later.

The diagnosis and examination institution 9 may also be constituted as a moving type of unit, where a general purpose medical appliance or examination device is mounted in a vehicle, train, ship, helicopter, or drone etc. that are dispatched to where patients exist. The control section 1 is capable of acquiring what medical institutions were visited, and what type of examination results were obtained, etc., from a server or the like that administers the diagnosis and examination institution 9. Conversely, the control section 9b is also capable of acquiring various data from the control section 1 in response to a request from a physician or the like associated with the diagnosis and examination institution 9. Naturally the server of the diagnosis and examination institution 9 may be the same as the control section 1, and some functions may also be shared.

In the diagnosis and examination institution 9, information of a user that has undertaken a medical examination etc., is transmitted to the communication control section 1a of the control section 1 using a data file DF2 file format. A data file DF2 is made up of acquired data RD2 and metadata MD2. Acquired data RD2 is a combination of data acquired by each device, and date and time, while the metadata MD2 includes device information of a device that has acquired the acquired data, and consultation result information etc. Other formats for the data file DF will be described later using FIG. 2A and FIG. 2B.

The DB section 9a of the diagnosis and examination institution 9 is an electrically rewritable non-volatile memory. The DB section 9a stores diagnosis results and examination results of the diagnosis and examination institution 9 for every individual ID. Also, the DB section 9a can store information associated with lifestyle habits of a user, and daily life guidance (lifestyle habit measures) for lifestyle habits, etc. Further, it is possible to store medicines etc. taken by the user.

Also, the DB section 9a stores genetic information for every patient, as required, and microbiome (one kind of normal bacterial flora) information, and accuracy may be improved using information that is stored in the DB section 9a at the time of consultation, and diagnosis, and at the time of inference. For example, these items of information may be stored by being simplified by categorization by a plurality of types, and with presence or absence information for normal bacterial flora, or specified genes. It is known that genetic information has an effect on cancers etc., and normal bacterial flora of a human form unique bacterial communities (bacterial flora, microbiomes) made up of bacterial species and with composition ratios that are different for each habitat, such as intraoral and enteric bacteria, and since resident flora is insusceptible from the outside, it is known that these types of differences play an important role in the health of people.

Also, a management database is provided in the DB section 9a for administering usage conditions for devices held by each medical institution. In recent years, with advances in the specialization of medical institutions, or advancements in primary care physician surgeries, patients with specified symptoms often go to the same clinic. There are cases where this clinic will not have examination devices or examination kits for other illnesses. Therefore, if it is made possible to also administer information relating to devices held at the medical institutions, it becomes possible to handle problems of labor involved with too many consultations, and infection risk etc. If this information can be made common with the DB section 8, it is possible to know, for each clinic, what clinic or hospital has supplementary functions, and it is possible to give appropriate advice to people visiting the hospital.

The control section 9b of the diagnosis and examination institution 9 is a controller (processor), and it is assumed to be an IT device comprised of a CPU (Central Processor Unit), that provides files and data etc. to other terminals by means of a server etc. or network provided in the diagnosis and examination institution 9, memory, an HDD (Hard Disc Drive) etc. However, the control section 9b is not limited to this structure, and in the case of constructing a small-scale system it is possible to be configured with something like a personal computer. The control section 9b has various interface circuits, and can cooperate with other devices, and various arithmetic control is possible using programs.

The display control section 9c of the diagnosis and examination institution 9 has a display control circuit and a communication circuit, and performs control of display on the display section 9f of the physician terminal 9e. The physician terminal 9e is a terminal used by physicians etc. at the diagnosis and examination institution 9, and may be connected to the control section 9b by wired communication such as an intranet in the hospital, and may be connected using wireless communication such as WiFi.

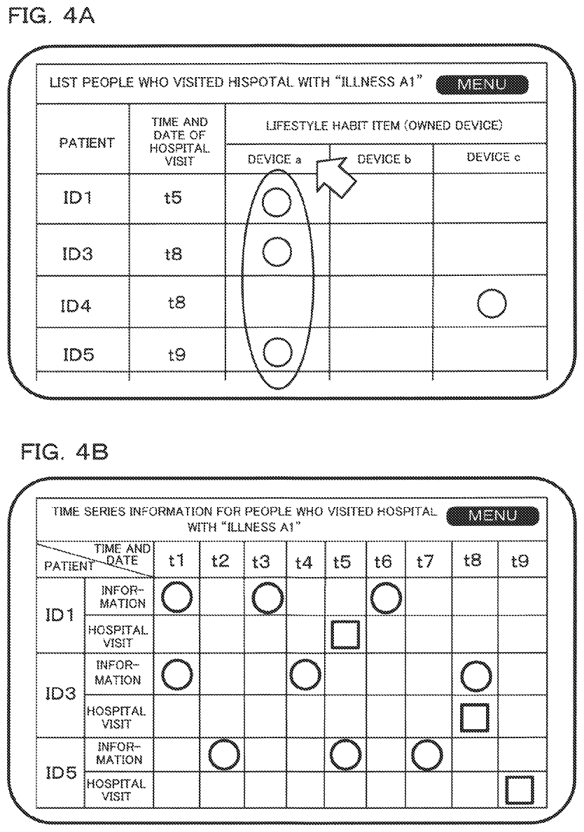

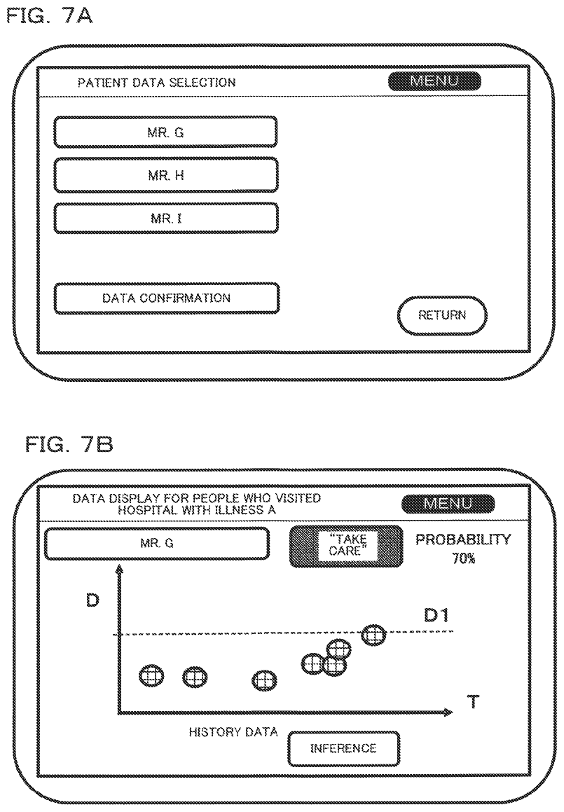

The display control section 9c functions as a display control section that performs list display of devices that are capable of acquiring a plurality of subjects (including object people) and chronological change of specified information at a plurality of times, on a display section (refer, for example, to FIG. 4A, FIG. 4B, S13 in FIG. 9A, etc.). The display control section 9c functions as a display control section that performs list display of chronological change of specified information at a plurality of times, on a display section (refer, for example, to FIG. 5A, FIG. 5B, S17 in FIG. 9A, etc.). The display control section 9c functions as a display control section (display control circuit) that displays diagnosis assisting information that has been acquired by inputting previous time series data of a patient to an inference model that has been generated as a result of a request to the learning request section, on the display section (refer, for example, to FIG. 7B, and S27 in FIG. 9B). It should be noted that the physician terminal 9e, and the control section 1, may possess functions of the display control section.

The physician terminal 9e may be a portable information terminal such as a smartphone or tablet, and may be a personal computer such as a desktop type or lap top personal computer. The display section 9f of the physician terminal 9e has a display, and displays information relating to health, relating to people who have visited the diagnosis and examination institution 9, as shown in FIG. 4A to FIG. 8. The display section 9f functions as a display section (display) that displays selection devices for data collection from a list of devices, in order to collect training data such that relationships between input and output of an inference model yield expected results (refer to FIG. 4A and FIG. 4B, for example).

Also, an operation section 9g is provided in the physician terminal 9e. The operation section 9g is an input interface for inputting operational information of the user. The operation section 9g has switches and buttons etc. for operation, and the front screen of the display section 9f constitutes a touch screen. The operation section 9g functions as an input section (input interface, terminal input section) for a physician to input symptoms of a specified patient (refer, for example, to S5 in FIG. 9A).

A control section 9h is a controller (processor), and comprises a CPU (Central Processor Unit) and memory etc. The control section 9h has various interface circuits, and can cooperate with other devices, and various arithmetic control is possible using programs. The control section 9h acts in cooperation with the control section 9b inside the diagnosis and examination institution 9, performs various display in accordance with operation using the operation section 9g, and executes various operations such as inference model request and inference operations etc. Also, the control section 9h is constituted by a processor having a CPU etc., as was described previously, and realizes functions of a device specification section, learning request section etc. Also, the control section 9h has a device specification section 9ha and a learning request section 9hb. Detailed display on the physician terminal 9e will be described later using FIG. 4A to FIG. 8.

The device specification section 9ha within the control section 9h specifies ID of a patient, and specified time series data that has been acquired by terminals owned by that patient, and by other devices, as shown in FIG. 4A, FIG. 4B, FIG. 5A, and FIG. 5B, for example. Specifically, the device specification section 9ha functions as a device specification section that specifies devices that are capable of acquiring previous time series data of the patient (refer, for example, to S13 and S17 in FIG. 9A). The device specification section performs setting from among devices that have been displayed on the display section (refer, for example, to FIG. 4A, FIG. 4B, and S13 in FIG. 9A).

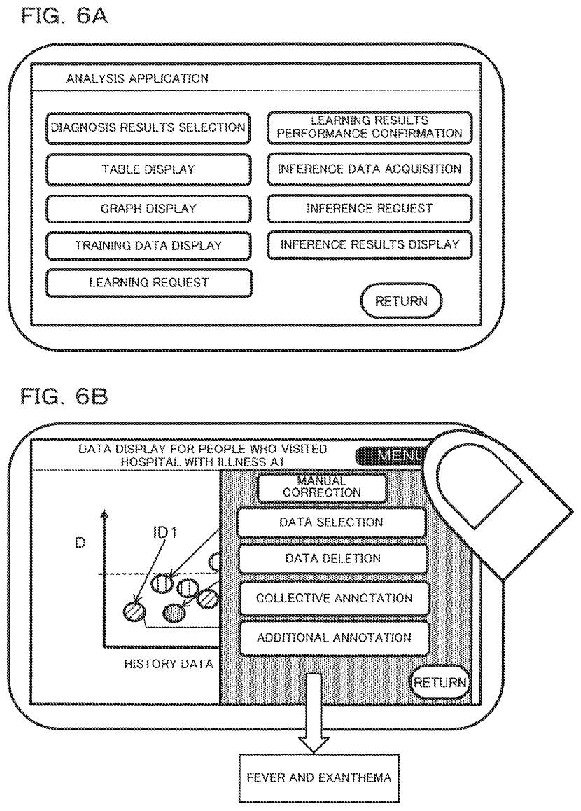

Also, training data is created using time series data and consultation information for other people having a similar device to the device owned by the patient (including devices capable of being used), as shown in FIG. 5A to FIG. 6B, and the learning request section 9hb within the control section 9h requests creation of an inference model using learning based on this training data. Specifically, the learning request section 9hb functions as a learning request section that makes time series data of another person having the same device as a device that has been specified, and consultation information, into training data, and requests learning (refer, for example, to S23 in FIG. 9B). The learning request section collects training data so that a relationship between input and output of the inference model generated in response to the learning request become such that collection data that has been collected by the same device is input, and information corresponding to symptoms of a patient that have been input by a physician is output. An inference model is obtained by learning using training data. Expected results of this inference model are that specified specimen information and bio-information is input, and diagnosis assisting information is output.

The terminal 4 is a portable information terminal that the user uses, and is a device for the user or a person related to the user to receive information that can be confirmed. As information there is health information, and facilities recommended in accordance with health status. The terminal 4 may be a smartphone or tablet PC, for example, and in this case it is possible to use a built in camera and microphone as an information acquisition section. Also, a linkable wearable terminal or other home appliance may be used as the terminal 4, and information may be acquired using a wearable terminal. Accordingly, the first device 2a, second device 2b, and terminal 4 may all be the same unit, or may be respectively dedicated devices. A terminal 4 that is linked to a wearable terminal may perform information acquisition and administration of information. Further, depending on conditions, functions possessed by the control section 1 may also be possessed by the first device 2a, second device 2b, third device 3, and terminal 4, and it is also possible to have a configuration whereby detection, control and information provision are performed in a distributed manner.

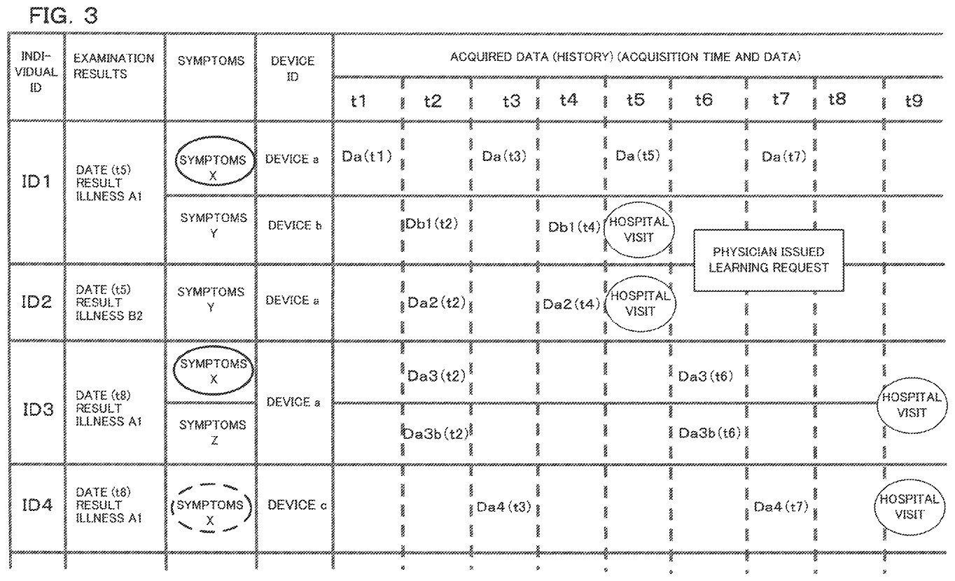

The database (DB) section 8 has an electrically rewritable non-volatile memory. The DB section 8 has a data history by ID list, and this list stores medical information, device ID, and history data for every time and date that examination data was acquired, for every individual ID (refer to FIG. 3). As was described previously, the ID determination section 1b receives examination data from the first device 2a etc. and diagnosis and examination institution 9 etc., and so the DB section 8 stores examination data separately for individual IDs. At this time, examination date, examination results, symptoms, examination device, acquired data, and date the diagnosis and examination institution 9 was visited, etc., are stored.

Also, the DB section 8 collects administration information such as for examination devices and examination kits owned by the medical institutions and examination institutions such as each clinic and hospital etc., and centralized control is possible. If it is known what devices exist at what locations, patients and physicians etc., can act to make determination based on accurate information, making it possible to handle problems of excessive infection risk and erroneous diagnosis. If patients and physicians etc. perform advice based on this type of facilities management, it is possible to access a storage section (DB) that stores owned equipment information for every examination and medical institution. The information provision section 1c can transmit effective information in which holding information and facility information is added, to eligible people. Specifically, in addition to examination data and profile information of the subject people, information dissemination according to owned equipment information for every examination and medical institution becomes possible.

Also, what type of examination is performed, and for what purpose an examination was performed, is also stored. The DB section 8 may organize data that has been acquired into the five W's, or 5W1H, namely, WHO, WHERE, WHEN, WHAT, WHY, and HOW, and store this organized data. Examination location (medical facility, examination institution, home, workplace, etc.) may also be stored. A storage example of data in the DB section 8 will be described later using FIG. 3.

Upon receipt of a request for generation of an inference model from the inference request section 1e within the control section 1, the learning request section 6 (refer to FIG. 1B) sends inference model specifications etc. to the learning section 5, and requests generation of an inference model conforming to the specifications. The learning request section 6 has a data classification and storage section 6a, a specification setting section 6d, a communication section 6e, and a control section 6f.

The control section 6f is a controller (processor) that controls within the learning request section 6, and it is assumed to be an IT device comprised of a CPU (Central Processor Unit), that provides files and data etc. to other terminals by means of a server etc. or network, memory, an HDD (Hard Disc Drive) etc. However, the control section 6f is not limited to this structure, and in the case of constructing a small-scale system it is possible to be configured with something like a personal computer. The control section 6f has various interface circuits, and can cooperate with other devices, and various arithmetic control is possible using programs.

The data classification and storage section 6a has an object type A image group 6b, and training data 6c is stored within this image group. The object type A image group 6b is an image group used at the time of generating an inference model in the learning section 5, and there are many image groups, being type A, type B, . . . , Training data 6c is generated based on this image group. Specifically, as shown in FIG. 5A and FIG. 5B, it is possible to draw a graph if examination data is plotted for every examination day, and it is possible to treat this graph as an image. It should be noted that although images are described here as easily intuitively understood, data does not have to be handled as images, and it is also possible to generate change in time series examination data, that is, a plurality of examination data groups in which examination dates and times, and examination data, has been collected, as training data. The data storage and classification and storage section 6a stores training data on the basis of a data history list stored in the DB section 8.

The specification setting section 6d sets what type of inference model will be generated, based on inference model specification that was determined by the inference model specification determination section 1d. Also, training data is generated from data stored in the history list of the DB section 8, so as to satisfy this specification.

The communication section 6e has a communication circuit for communicating with the control section 1 and the learning section 5. A request for generation of an inference model is received from the control section 1 by means of this communication section 6e, and generation of an inference model is requested to the learning section 5.

The learning section 5 has an input output modeling section 5a, and an inference model is generated by machine learning or the like in accordance with specifications from the learning request section 6. The input output modeling section 5a has a specification checking section 5b. This specification checking section 5b determines whether or not specifications received from the learning request section 6, and an inference model that has been generated by the input output modeling section 5a, match. Specifically, the specification checking section 5b is for stipulating not only input output relationships, but also stipulating time and energy taken by inference by the inference model, and circuit structure etc., and a method of learning so as to perform learning in line with “required specifications”.

An inference model is generated by learning relationships between acquired information such as bio-information that has been acquired and biopsy information, and diseases, that is, by learning relationships between acquired information and diagnosis and treatment departments and sections. The input output modeling section 5a has an input layer, a plurality of intermediate layers, and an output layer, similarly to the inference engine 7, and generates an inference model by obtaining strengths between connections of neurons of the intermediate layers by learning.

When generating this type of inference model, the learning request section 6 extracts change patterns of examination data, that has been acquired from an examinee by using an examination device etc., for a specified time width, inputs this change pattern that has been extracted to the inference engine 7, makes health advice that should be output at a later timing after the time the examinee was examined into annotation information, and generates training data. Then, the learning section 5 generates an inference model by performing learning using this training data.

Also, if the learning section 5 performs learning using an examination data array after examination, attending a hospital, and taking medicine, it is also possible to generate an inference model that is capable of performing future expectation advice for the effects of lifestyle habit improvements, medical treatment, and taking of medicine. In this case, time of examination, attending hospital and taking medicine is made a start point, and time series data after that is used. In the case of advice for examination, attending hospital and taking medicine etc., time series data before that is used.

Here, description will be given of deep learning, as one example of learning that is performed by the learning section 5. “Deep Learning” involves making processes of “machine learning” using a neural network into a multilayer structure. This can be exemplified by a “feedforward neural network” that performs determination by feeding information forward. The simplest example of a feedforward neural network should have three layers, namely an input layer constituted by neurons numbering N1, an intermediate later constituted by neurons numbering N2 provided as a parameter, and an output later constituted by neurons numbering N3 corresponding to a number of classes to be determined. Each of the neurons of the input layer and intermediate layer, and of the intermediate layer and the output layer, are respectively connected with a connection weight, and the intermediate layer and the output layer can easily form a logic gate by having a bias value added.

While a neural network may have three layers if simple determination is performed, by increasing the number of intermediate layer it becomes possible to also learn ways of combining a plurality of feature weights in processes of machine learning. In recent years, neural networks of from 9 layers to 15 layers have become practical from the perspective of time taken for learning, determination accuracy, and energy consumption. Also, processing called “convolution” is performed to reduce image feature amount, and it is possible to utilize a “convolution type neural network” that operates with minimal processing and has strong pattern recognition. It is also possible to utilize a “recursive neural network” (fully connected recurrent neural network) that handles more complicated information, and with which information flows bidirectionally in response to information analysis that changes implication depending on order and sequence.

In order to realize these techniques, it is possible to use conventional general purpose computational processing circuits, such as a CPU or FPGA (Field Programmable Gate Array). However, this is not limiting, and since a lot of processing of a neural network is matrix multiplication, it is also possible to use a processor called a GPU (Graphic Processing Unit) or a Tensor Processing Unit (TPU) that are specific to matrix calculations. In recent years a “neural network processing unit (NPU)” for this type of artificial intelligence (AI) dedicated hardware has been designed to be capable being integratedly incorporated together with other circuits such as a CPU, and there are also cases where they constitute some parts of processing circuits.

Besides this, as methods for machine learning there are, for example, methods called support vector machines, and support vector regression. Learning here is also to calculate discrimination circuit weights, filter coefficients, and offsets, and besides this, is also a method that uses logistic regression processing. In a case where something is determined in a machine, it is necessary for a human being to teach the machine how determination is made. With this embodiment, determination of an image adopts a method of performing calculation using machine learning, and besides this may also use a rule-based method that accommodates rules that a human being has experimentally and heuristically acquired.

The inference engine 7, similarly to the input output modeling section 5a of the learning section 5, has an input layer and an output layer, and a neural network. The inference engine 7 performs inference using an inference model that has been generated by the learning section 5. For example, the inference engine 7 is input with time series bio-information that has been measured by the first device 2a etc., and obtains, for example, examination institutions and medical institutions suitable for performing examination and treatment etc. of the health status of the user, by means of inference. Also, inference such as when a consultation will be given at a medical institution may also be performed based on time series bio-information.

In this way, besides the retrieval section 1f searching the DB section 8, the control section 1 may also provide information relating to illness of the user utilizing the inference engine 7. The inference engine 7 performs inference relating to illness using an inference model that has been generated by the learning section 5. This inference model is generated by learning relationships between acquired information, such as bio-information that has been acquired and biopsy information, and diseases. In this manner it is possible for the control section 1 to also output guide information to be presented using inference by the inference engine 7.

If guidance is issued for an illness or the like with a single determination, based on acquired information that has been obtained only once as a result of the control section 1 searching or by inference, there is a possibility of unnecessarily bringing medical information into someone's life, which in itself might hinder someone living a healthy and stress free life. Therefore, accuracy may be improved using history of acquired information for a plurality of times (time series information).

Next, file structure of a data file DF that can be used as training data for learning will be described using FIG. 2A and FIG. 2B. FIG. 2A shows file structure of a data file DF3 that can be used as first training data for learning. This data file DF3 has acquired data RD3a, RD3b, and RD3c that has been acquired by examination devices at different dates and times, and metadata MD3 of these acquired data. As acquired data, although only three are described in FIG. 2A, this number would increase accordingly, for example, if the number of examinations increases. Also, as the metadata MD3, information on devices that were used in examination, consultation results, and ID identifying the user who underwent examination, etc., are stored. It is also possible to describe classification of people that have added annotation to the metadata, and how specialists have been involved, and IDs etc. specifying individuals and organizations who have performed annotation.

FIG. 2B shows a case where data files are gathered together in a folder format. With the example shown in FIG. 2B, consultation results for patient A are gathered together in a folder. Identification data IDa4 for specifying patient A, and data for storing consultation results MDRe4, are stored in this folder. Also, data files DF4a, DF4b, and DF4c are stored for respectively acquired data. The format of these data files is substantially the same as that of the data file DF1 that was shown in FIG. 1A, and so detailed description is omitted. It should be noted that although only three data files DF4a etc. are described in FIG. 2B, this number would increase accordingly, for example, if the number of examinations increases.

Next, history data etc. that is stored in the DB section 8 will be described using FIG. 3. This history data is created for every individual ID for identifying users individually. The history data stores examination results, symptoms, device ID, and acquired data for every ID. As examination results, information relating to illness based on examination time and data and consultation results is stored. As symptoms, symptom names are entered, examination device ID is stored for every symptom, and acquired data that was acquired using that examination device is stored for every date. Further, the day a physician was visited at a diagnosis and examination institution 9, such as a hospital, is stored.

With the example of history data shown in FIG. 3, a user having ID1 acquires examination data Da(t1), Da(t3), Da(t5) and Da(t7) for dates and times t1, t3, t5, and t7, using device a that is capable of examination for symptoms X. Also, the user having ID1 acquires examination data Db1(t2), and Db1(t4) for dates and times t1 and t4, using device b that is capable of examination for symptoms Y. For ID1, after visiting hospital at date and time t5, a physician determines that they are suffering from illness A1.

Also, the user having ID2 acquires examination data Da2(t2), and Da2(t4) for dates and times t2 and t4, using device a that is capable of examination for symptoms Y. Then, for ID2, after visiting hospital at date and time t5, a physician determines that they are suffering from illness B2.

Also, on time and date t5, when ID1 and ID2 visited the hospital, a physician requested learning using history data for both users. That is, with ID1 as an example of suffering from illness A1, and ID2 as an example of not suffering from illness A1, there is time series examination data, and further, generation of an inference model is requested to the learning section 5, with time series data that was examined with similar devices as training data for learning.

Also, for ID3 and ID4 also, data is similarly acquired using device a and device c, and stored in the DB section 8. Both users visited the hospital on time and date t8, and saw a physician. For ID3 there is data from device a, and there are symptoms X. On the other hand, for ID4 there is only data for device c, and there are symptoms X. As was described previously, the physician requested learning at time and date t5, and acquired an inference model. By inputting time series examination data for ID3 and time series examination data for ID4 to this inference model, it is possible to infer whether or not there is illness A1. The physician can perform diagnosis as to whether or not there is illness A1 by referring to this inference result.

Next, display on the display section 9f of the physician terminal 9e will be described using FIG. 4A and FIG. 4B. It should be noted that the examples shown in FIG. 4A and FIG. 4B are for a case where ID1 and ID2 etc., having the history data that was shown in FIG. 3, see a physician.

FIG. 4A shows a list display for a patient who has visited hospital with illness A1. There are cases when a physician, when determining an illness, would like to confirm examination devices used by the patient who is suspected of suffering from the illness, and history data (refer to S11 and S15 in FIG. 9A). FIG. 4A shows list display of hospital visit time and date for every patient that has visited the hospital with the same illness, and devices being used by that patient. With the example of FIG. 4A, it is shown that patient ID1, patient ID3, and patient ID5 have history data that has been examined using device a, while patient ID4 has history data that has been examined using device c.

The display in FIG. 4A is performed by means of transmitting data, so that the control section 9b of the diagnosis and examination institution 9 searches for appropriate data from among data that is stored in the DB section 9a, and the display control section 9c can display search results on the physician terminal 9e. That is, the display control section 9c functions as a display control section (display control circuit) that is capable of list display of devices that are capable of acquiring a plurality of subjects (subject people) that have been determined to have a specified illness (illness A1), and chronological change in specified information at a plurality of points in time. It should be noted that besides the control section 9b performing search of data, the control section 1 may also search data that is stored in the DB section 8. The same also applies to the case of FIG. 4B which will be described later.

FIG. 4B shows time series information of a patient who has been to the hospital with illness A1. There are cases when list display of patients who have visited the hospital with illness A1 continues, as shown in FIG. 4A, and a physician would like to confirm time and date information of examinations for those patients, and when they visited the hospital. FIG. 4B is a list display showing examination time and date and hospital visit time and date for patients who visited the hospital with the same illness.

The display in FIG. 4B is performed by means of data transmission, so that the control section 9b of the diagnosis and examination institution 9 searches for appropriate data from among data that is stored in the DB section 9a, and the display control section 9c can display search results on the physician terminal 9e. That is, the display control section 9c functions as a display control section (display control circuit) that is capable of list display of chronological change in specified information at a plurality of points in time when a specified illness (illness A1) has been determined.

Next, data display for patients who have visited the hospital will be described using FIG. 5A and FIG. 5B (refer, for example, to S15 and S17 in FIG. 9A). As shown in FIG. 4A and FIG. 4B, a list of patients who have been to the hospital with illness A1 (with owned terminals and hospital visit time and date information) is displayed on the display section 9f of the physician terminal 9e. In a case where there are patients who have made a hospital visit at the hospital, it is useful if a physician can see time series change in the examination data of those patients on their terminal. Therefore, with this embodiment, if graph display is selected on a menu screen or the like of the physician terminal 9e (refer to FIG. 6A and FIG. 6b), the graphs shown in FIG. 5A and FIG. 5B are displayed.

FIG. 5A shows examination data D for patients that have undertaken a consultation for illness A1, up until time t9. In the graph of FIG. 5A, the horizontal axis shows date and time of hospital visit, and the vertical axis shows examination data D. Circles in the graph represent examination data D for patients ID1, ID3 and ID5. On the right side of FIG. 5A, icons for “consultation”, “no consultation”, and “both” are displayed. FIG. 5A shows history data for patients who have been diagnosed with illness A1, and so the icon for “consultation” is shown in inverse display.

In the display state of FIG. 5A, if the “no consultation” icon is touched, the “no consultation” icon is shown in reverse display, as shown in FIG. 5B, and a graph for “people who have not visited the hospital, with illness A1” is displayed. In the graph of FIG. 5B also, the horizontal axis shows date and time of hospital visit, and the vertical axis shows examination data D. Circles in the graph represent examination data D for patients ID2, ID4 and ID6. ID2, ID4, and ID6 are people who have been to the hospital but were not diagnosed with illness A1. In this state, if the “both” icon is touched, history data of both patients that have been diagnosed with illness A1 and patients who have not been diagnosed with illness A1 is subjected to graph display.

Using the displays such as FIG. 5A and FIG. 5B, it is possible to visually confirm time series change for this case, and by displaying in combination with other information there is also a high possibility that a physician will reference this graph information at the time of performing diagnosis and treatment. This display can be achieved with only data collection and the making of a graph, and there are cases where some knowledge or information is obtained without creating training data, or without waiting for inference. A data collection device having an input section for a physician to input information relating to symptoms of a patient, a device specification section for specifying a device that is capable of acquiring previous time series data of the patient, and a data collection section for collecting collection data using a device of a separate person having the same device as the device that was specified, can collect information that will constitute reference material for a physician at the time of diagnosis and treatment, as has been described above. Also, if the results of this collection are displayed and a physician is made aware, it will useful in health maintenance information of many people.

Further, a training data collection device having a learning request section that requests learning with consultation information made into training data can obtain more objective information. By using training data that has been collected by this training data collection device, if a high reliability inference model is created it becomes possible to share the awareness and knowledge of physicians, it is possible to standardize diagnosis methods throughout the world, and it becomes possible to present highly precise health recovery measures and health maintenance measures without being dependent of the experience of physicians. However, if all unorganized information is used as training data it will not be possible to obtain a high reliability inference model, and so it should be made possible to choose use and disuse of data.

If a time axis for data that has been collected can be seen, then in a case such as infectious disease it is possible to determine in what area an epidemic started etc. In a case where there are a lot of people with a fever on a specified day, confirming whether or not infectious disease has spread from overseas etc. on that day is made a criterion when performing determination. Also, with time axis display based on time of hospital visit, it becomes easy to confirm processes etc., leading to a hospital visit based on changes in cases (symptoms) specific to that illness, in accordance with when the patient became aware that they were coming down with an illness, or when people who know or have seen the patient recommended going to hospital.

Also, if collected data is displayed on a time axis based on points in time where there is changed in specified data (for example, time when fever intensified), changes in symptoms specific to that illness will be known, and this data that has been collected will constitute base data useful for diagnosis. Naturally, if a device is a portable terminal, history such as behavior history of that person, and internet access etc. is also stored as information (as a system that would also include information on the cloud), which means that trends of change in symptoms are grasped from further analysis of behavior information, making it possible to suppress advancement of an illness, and incorporate into information for improving health.

With the display in FIG. 5A and FIG. 5B, the control section 9b of the diagnosis and examination institution 9 searches for appropriate data from among data that is stored in the DB section 9a, and data is transmitted to so that the display control section 9c can display search results on the physician terminal 9e. That is, the display control section 9c functions as a display control section that is capable of list display of chronological change in specified information at a plurality of points in time when a specified illness (illness A1) has been determined.

The history data shown in FIG. 5A is data of people who have been diagnosed with any illness A1, and so for the data of these people it is possible to create training data to which annotation of “illness A1” has been attached. Also, the history data shown in FIG. 5B is data of people who have not been diagnosed with any illness A1, and so for the data of these people it is possible to create training data to which annotation of “not illness A1” has been attached (refer, for example, to S15 and S17 in FIG. 9A). File format for this training data for learning may be appropriately selected, such as data files DF1, DF2, DF3, DF4.

If training data can be created, the physician terminal 9e can request generation of an inference model suitable for illness A1, to the learning section 5, by means of the inference request section 1e of the control section 1, and the learning request section 6 (refer to S23 in FIG. 9A, for example). It should be noted that a request may be issued directly from the diagnosis and examination institution 9 to the learning request section 6 and learning section 5.

Next, menu screens of the physician terminal 9e will be described using FIG. 6A and FIG. 6B. If the physician terminal 9e is activated, a terminal menu is displayed. If the analysis application is launched on the terminal menu, the screen of “analysis application” shown in FIG. 6A is displayed (refer to S7 in FIG. 9A). Icons of “diagnosis results selection”, “table display”, “graph display”, “training data display”, “learning request”, “learning results performance confirmation”, “inference data acquisition”, “inference request”, inference results display”, “return”, and “MENU”, are displayed on this analysis application screen.

If “diagnosis results selection” is selected on the screen of the analysis application (refer to S11 in FIG. 9A, for example), a list of illnesses is displayed, and it is possible to select the name of an illness from within that list. For example, if illness A1 is selected, a list display of patients who have visited the hospital, such as shown in FIG. 4a, is displayed. In a state where diagnosis results have been selected, if “table display” is selected (refer, for example, to S15 in FIG. 9A), time series information such as shown in FIG. 4B is displayed as a table.

Also, if “graph display” on the menus screen of FIG. 6A is selected (refer, for example, to S15 in FIG. 9A), history data such as shown in FIG. 5A and FIG. 5B is displayed as a graph. At the time of the graph display of FIG. 5A and FIG. 5B, if “MENU” at the upper right is touched, icons such as shown in FIG. 6B are displayed. If “manual correction” is touched in this display state, it becomes possible to edit data. If specified data is selected using “data selection”, and “data deletion” is touched, that data will be deleted.

If “collective annotation” is touched in the state of FIG. 5A where patients who have visited hospital with illness A1 have been selected, training data to which annotation of “there is illness A1” has been attached are collectively generated. Also, if “additional annotation” is touched, it is possible to add annotation that will be attached to the training data. It is possible to group symptoms that are seen as being common to patients who have visited the hospital with illness A1, and add as annotation. With the example shown in FIG. 6B, “fever and exanthema” is appended as annotation. This annotation is preferably text input by operating the operation section 9g of the physician terminal 9e.

Returning to FIG. 6A, if “training data display” is touched (refer to S15 in FIG. 9A, for example), a physician selects data of FIG. 5A and FIG. 5B etc., and data is displayed as training data. If “learning request” is touched (refer to S21 in FIG. 9B, for example), the physician terminal 9e requests generation of an inference model to the learning section 5 using the training data that was displayed by “training data display”. If “learning results performance confirmation” is touched (refer to S21 in FIG. 9B, for example), then when learning has been requested, performance, for example reliability, of an inference model that the learning section 5 has generated is evaluated. This evaluation is performed by preparing data for evaluation, for example, inputting this data for evaluation to the inference model, and performing evaluation based on the output result. If satisfactory results are obtained as a result of having performed learning results performance confirmation, an inference model is acquired.

In FIG. 6A, if “inference data acquisition” is touched (refer to S25 in FIG. 9B, for example), inference data for input to the inference model is acquired. For example, there are cases where, in the example of FIG. 3, at the time of time and date t9, when ID3 and ID4 visited the hospital, a physician inputs history data for ID3 and ID4 up to that point to the inference model, and inference is performed for an illness. This inference data is previous history data for a patient who was the subject of a consultation in this way.

If “inference request” in FIG. 6A is touched (refer to S25 in FIG. 9B, for example), inference data that has been acquired is input to the inference model, and output of inference results is requested. If the diagnosis and examination institution 9 has an inference engine, then the diagnosis and examination institution 9 is made the destination for the request for inference. In the event that the diagnosis and examination institution 9 does not have an inference engine, the request may be sent to the control section 1. Naturally, if the physician terminal 9e has an inference engine inference may be performed within the physician terminal 9e. Detailed screens for this inference request will be described later using FIG. 7A and FIG. 7B. If “inference results display” is touched, inference results are displayed.

Next, screens for inference request will be described using FIG. 7A and FIG. 7B. There are cases where a physician wishes to obtain inference results using an inference model for change in symptoms in the future from previous history data of a patient (refer, for example, to S25 in FIG. 9B). In this case, the physician touches “inference request” on the screen of FIG. 6A. If “inference request” is touched, then first a screen for patient data selection is displayed, as shown in FIG. 7A. With the example shown in FIG. 7A, a patient name, such as “Mr. G” is displayed.

If a patient name is selected by touching the patient name in FIG. 7A, then history data of the patient who has been selected is subjected to graph display, as shown in FIG. 7B. It is possible for the physician to understand previous data of the patient using the graph display, and further, if the physician wants to perform prediction for the future they touch “inference” at the bottom of the screen. If “inference” is touched, inference is performed and inference results are displayed within the screen. With the example shown in FIG. 7B, a probability of contracting illness A1 is “70%. A physician can get diagnosis results by referencing these inference results.

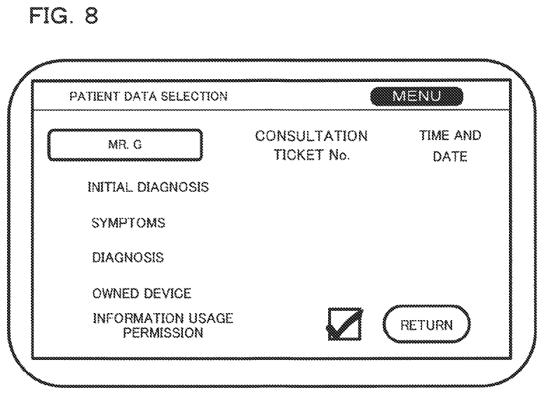

Next, a screen for performing diagnosis input will be described using FIG. 8. If the screen for performing diagnosis input is opened from the terminal menu screen (refer to S1, S3, and S5 in FIG. 9A, for example), and a patient name is selected, the screen for diagnosis input of FIG. 8 is displayed. The physician provides input of symptoms for a specified patient on this screen for diagnosis input. With the example of FIG. 8, “Mr. G” is selected as a patient name. If data input has already been performed to this screen, consultation ticket No. for Mr. G, and time and date, are displayed. Also, if data has been input for initial diagnosis, symptoms, diagnosis results, and owned device, that data is displayed, and the physician performs input to the physician terminal 9e for items that can be entered when there has not yet been input. Also, regarding use of individual information of a patient, in the event that permission has been obtained from the patient a check box at the lower part of the screen is checked. In FIG. 8, permission has been obtained and so a check mark is affixed.

Next, operation of the control section 9h in the physician terminal 9e will be described using the flowcharts shown in FIG. 9A and FIG. 9B. This flow is realized by the control section 9h within the physician terminal 9e cooperating with the control section 9b within the diagnosis and examination institution 9 to control each section within the diagnosis and examination institution 9 and the physician terminal 9e.

If the power supply of the physician terminal 9e is turned on and the flow shown in FIG. 9A is commenced, first, a terminal menu is displayed (S1). Here, the control section 9h displayed the menu screen on the display section 9f. As menu display, “diagnosis results input”, “launch analysis application”, and other functions etc., and items that can be operated, are displayed as icons.

Once menu display has been performed, it is next determined whether or not determination results will be input (S3). Here, the control section 9h performs determination based on whether or not “diagnosis results” on the menu screen has been subjected to a touch operation.

If the result of determination in step S3 is that determination results will be input, input is performed (S5). Here, the control section 9h displays an input screen or diagnosis results shown in FIG. 8 on the display section 9f. As was described previously, with the diagnosis results input screen it is possible for the physician to input diagnosis results of the patient etc. using the operation section 9g. Specifically, in this step the physician performs input of symptoms of a specified patient. Besides this, input of examination data results at the time of consultation etc. is also performed.

If input has been performed in step S5, or if the result of determination in step S3 is that there is not input of diagnosis results, it is next determined whether or not to launch the analysis application (S7). Here, the control section 9h performs determination based on whether or not “analysis application” on the menu screen has been subjected to a touch operation.

If the result of determination in step S7 is not to launch the analysis application, other functions are executed (S9). Here, the control section 9h performs other functions, such as, for example, device loan, device registration, signed agreement for a patient etc., entry and confirmation of normal clinical records, etc. Once these other functions have been executed, step S1 is returned to.

If the result of determination in step S7 is to launch the analysis application, it is determined whether or not there is a list of diagnosis results is confirmed (S11). Here, the control section 9h first displays the menu screen of the analysis application shown in FIG. 6A on the display section 9f. Icons corresponding to various items are displayed on the menu screen, as was described previously, and so in this step the control section 9h determines whether or not the “diagnosis results selection” icon has been selected.

If the result of determination in step S11 is that diagnosis results list conformation has been selected, patients and devices are displayed in accordance with diagnosis results, and device selection is performed (S13). Here, the control section 9h shows list display of patients who have visited the hospital with a specified illness, such as was shown in FIG. 4A. This list display displays examination devices that the patients own, or that the patients are capable of using. A physician can select examination devices that have been displayed. For example, in the case where, in FIG. 4A, a lot of patients who have visited the hospital because of illness A1 have (or are capable of using) device a, it is possible to select device a.

If selection of devices has been performed in step S13, or if the result of determination in step S11 was that diagnosis results list confirmation was not selected, it is next determined whether or not to display table display, a graph, and training data (S15). Here, the control section 9h determines whether or not any one of the “table display”, “graph display”, and training data” that were shown in FIG. 6A has been selected.

If the result of determination in step S15 is that any display such as table display has been selected, it is made possible to display, confirm and choose data by patient, corresponding to the selected device (S17). Here, the control section 9h displays time series information of people who visited the hospital with a specific illness, that was shown in FIG. 4B, history data of people who visited the hospital with a specific illness, that was shown in FIG. 5A and FIG. 5B, and history data of people who did not visit the hospital with a specific illness, on the display section 9f. For example, as shown in FIG. 4A, a lot of data, that has been examined using specified terminals that patients who have visited hospital for a specified illness (illness A1) either own or are capable of using, can be collected (refer to FIG. 5A). This data can be used as training data for determination of patients having a specified illness. Also, it is possible to collect a lot of data that has been examined using specified devices, for patients that have not visited hospital for a specified illness. This data can be used as training data for determination of patients who do not have a specified illness. Input and output relationships of an inference model that is obtained from results of learning using this this data is such that collected data that has been collected using a similar device is input, and information corresponding to symptoms of a patient that has been input by a physician is output. Data is collected so as to obtain this type input output relationship for the inference model.

Also, in step S15, it is possible to confirm training data used in a request for learning by selecting “training data” on the menu screen of the display section 9f. Also, when history data (training data) has been displayed, and when MENU is touched, it is possible to select data and perform choosing of data deletion etc. (refer, for example, to FIG. 6B). In this way, it is possible to create training data, for generation of an inference model for determination of a specific illness, by using these icons.

If the processing of step S17 has been executed, or if the result of determination in step S15 is that table display etc. is not performed, it is next determined whether to perform a learning request, or confirm learning results (S21). Here, the control section 9h determines whether any of “learning request”, or “learning results performance confirmation”, that were shown in FIG. 6A have been selected.

If the result of determination in step S21 is learning request or learning results confirmation, learning is requested with training data that has already been selected, or results are acquired (S23). Here, it is possible to request the learning section 5 so as to generate an inference model using training data that was selected in step S17. An inference request is executed, for example, by selecting the “inference” icon as shown in FIG. 7B.

Also, in a case where learning has been requested to the learning section 5 in step S23, and an inference model has been generated, results for performance and reliability etc. of that inference model are acquired, and displayed. Performance and reliability of the inference model are determined by calculating a LOSS value, etc., and basing performance and reliability on this LOSS value. Data that has been prepared in advance for evaluation is input to the inference model, and LOSS value is a value that represents a degree to which inference results at this time match with results for data that has been prepared in advance. This evaluation of performance and reliability is performed for devices that have an inference engine. In the event that performance and reliability are less that a specified level as a result of evaluation, training data is created again, and restarting of device selection etc. is performed, and an inference model is created again using the learning section 5.

In the event that it was possible to generate an inference model of high reliability in step S23, it is made possible to use the inference model on a specified server or the like, and it is made possible to use at many medical institutions, and a general user confirming their own status is made available. As a result, it is possible to appropriately perform arrangement of ambulances, and it becomes possible to reduce illness at medical institutions etc., and to resolve issues with medical sire being busy. It is possible to prevent people being infected, and infecting other people, on the way to visiting a hospital, and at medical institutions that are to be visited. Also, an ID is provided in this inference model, and it may be made possible to know what inference model determination has been made with. If infinite similar models appear, those that are of poor quality may propagate excessive uncertainty, or there is a possibility that patients in need of urgent care will be seen too late. It is desirable to specify AI using ID, and this is also useful in authenticity of AI.

Also, in a case where an inference model (AI) has been generated with physician's awareness and training data choices, it is desirable to clarify who created that inference model. As a result of clarifying the creator, outcome at the time of difficult creation with AI is that the efforts of that physician are widely acknowledged, and it is possible to take steps such as providing remuneration for that effort. Also, it becomes possible to easily prevent malicious AI appearing on the market. This type of AI determines what type of data is required, and so data that is suitable for that AI may perform schemes that can clarify those conditions. Specifically, at the time of acquiring data directed to that AI, an application that starts data acquisition by designating that AI is installed in a device, and it should be made possible to enter assumed AI information (ID etc.) as metadata of a data file of acquired data, such as in FIG. 2A.

If inference model results have been acquired in step S23, or if the result of determination in step S21 was that there was no learning request and learning results confirmation, it is next determined whether inference data has been acquired, inference has been requested, and inference results acquired (S25). As was described previously, when a physician has consulted with a patent, history data of that patient is input to an inference model, and there are cases where it is desired to obtain inference results relating to illness. In this step, inference that used an inference model is performed. In this step, the control section 9h determines whether or not any one of “inference data acquisition”, “inference request” and “inference results display” that were shown in FIG. 6A have been selected.

If the result of determination in step S25 is inference data acquisition etc., then next, a request to download an inference model, and results acquisition, are performed (S27). Here, the control section 9h requests download of an inference model that satisfied the specified performance and reliability in step S23, to the learning section 5. Also, history data of a patient that inference is desired for is acquired, this history data is input to the inference model that has been downloaded, and inference results are acquired. Inference results that have been acquired are displayed on the display section 9f (refer to FIG. 7B, for example).

If inference results have been acquired on steps S27, it is next determined whether or not to return (S29). Here, the control section 9h performs determination based on whether or not “return” in the menu screen (refer to FIG. 6A, FIG. 6B, FIG. 7A, and FIG. 8) has been selected. If the result of this determination is that return has not been selected, processing returns to step S11, while if return has not been selected processing returns to step S1.

In this way, depending on operation of the control section of the physician terminal 9e, a physician inputs information relating to symptoms of a patient (S5), a device that is capable of acquiring time series data of the patient is specified (S13), and time series data and consultation information of another person having a similar device to the specified device is made into training data and a learning request is issued (S27). As a result, if new symptoms have occurred it is possible to generate an inference model using examination data of other people that use a similar device, and consultation information of those other people. If this inference model is used, then even if new symptoms occur it is possible to use this information for other people as reference information when performing precise consultation. Also, if it is known what person and at what time this data is for, it does not need to be time series data. For example, with fever etc. there will be cases of sudden outbreak, and time series data could constitute training data that can be used without being analyzed.

Also, when a physician consults with a patient, after an inference model has been generated, in a case where it is desired to perform inference relating to illness based in history data of the patient (S25 Yes), then history data of the patient is input to the inference model, and inference results are obtained (S27). The physician can then diagnose the symptoms of the patient referencing these inference results.

In this way, an inference model for what type of consultation results, and what diagnosis results mean, is created from health related data, and if it is made possible for people to appropriately use the services and systems etc. that they want to use, then by inputting data that is acquired on a daily basis from first to third devices (refer to FIG. 1A) to this inference model it becomes possible to utilize in many diagnosis support and health management situations. This approach came about with the background of it having become possible to acquire health related information of many people from various monitoring devices (for example, the first to third devices in FIG. 1A) throughout the world, and useful information has become readily accessible using information terminals, as a result of connecting together various devices on the Internet stemming from the creation of the Internet of Things (IoT) using advancements in IT technology.

Using these technologies, it has become possible to create tools where monitoring devices (refer to the first to third devices in FIG. 1A, for example) are used to encourage health consciousness of each individual, and confirming the necessity for hospital visits etc. As a result of patients going to the trouble of receiving a consultation despite suffering from an illness, and a physician using various examination information and inquiries, there are important diagnosis results that take time to obtain, and so if an inference model that has been created by means of the above described process is referenced at the time of diagnosis by another physician it is also possible to address problems of lack of physicians and infectious diseases of recent years.

It should be noted that the processes of steps S11 to S29 are performed by the control section 9h in association with the physician, but it is also possible for a computer to perform specified programs in a routine-based manner. Specifically, a physician may also select an icon on the menu screen of the physician terminal 9e (refer to FIG. 6A), and each step may be sequentially executed sequentially automatically.

Next, another example of operation of the control section will be described using the flowcharts of FIG. 10A and FIG. 10B. The previously described operation of the control section was operation of the control section 9h of the physician terminal 9e. Specifically, the example of control section operation was execution of operation such as autonomous display by the control section 9h of the physician terminal 9e. Conversely, this other example of operation of the control section is execution by a server side control section 1 of operations such as receiving a request from the physician terminal 9e and the diagnosis and examination institution 9, and display etc. Specifically, execution of operations such as display of the physician terminal 9e using the cloud. It should be noted that function as the cloud may be execution by the control section 9b of the diagnosis and examination institution 9, as well as by the control section 1.

If the flow shown in FIG. 10A is commenced, first, data from each device is collected and made into a database (SB) (S31). Here, the control section 1 collects examination data from examination devices such as the first device 2a, second device 2b, and third device 3, and stores this data in the DB section 8.

Collection of data is commenced in step S31, but the time for commencing data collection may be instructed by the physician, and data collection may be started automatically by each user themselves (a patient, candidate patient, or device user), or a device itself being used by each user, noticing events. Also, a device may commence such data accumulation at the time equipment is introduced or purchased, or at the time of a specified service contract. In a case where a device automatically performs data collection, at the time that data that has been input using a data input section is other than expected data, there may be a handling determination section to designate the fact that data is other than expected data, and collect this designated data. If there is a system having this handling determination section, it is possible to make a system that is capable of inferring causes of abnormality, even under conditions where a physician cannot intervene.

Data that has been collected by the control section 1, or time series data groups, are transmitted to the learning section 5 by means of the inference request section 1e and learning request section 6 in step S61, which will be described later, and generation of an inference model is requested. This inference model may be possessed by the control section 1, or may be possessed by each device (for example, the first to third devices etc.). By inputting health related data and monitored data of a user to this inference model it becomes possible to infer health status, and it is possible for each user to ascertain their own health status from the results of this inference.

Since diagnosis results of the physician are reflected in this inference model, highly reliably and accurate inference becomes possible. As required, what physician created the inference model, and what type pf specification the inference model has may also be displayed together with inference results on the user terminal. Many physicians will have similar awareness, and there are physician's non-profit organizations, and so there is a possibility of creating similar inference models while adding unique schemes, and while adding information such as what data will be used etc. An inference model may be selected automatically from features of input (monitored) data, and it may also be made possible for users to select popular models, and to publish evaluation on the Internet.

It is also possible to have a system having an inference request section that performs learning requests for inference models in accordance with an inference system, with data that is other than expected collected data as training data. This system handles data of other devices where other similar events are happening, including effective abnormalities of the user and any kind of problem with devices, or of other devices of similar conditions, as big data, and enables collection of information for determining what types of conditions those conditions are (for example, is it something commonly occurring, is it something happening in a group, is it an isolated accident?). An unknown event for any person intensifies anxiety, but it is also possible to collect information that reduces anxiety, and in a case where there is an emergency it is possible to perform confirmation and determination from similar data that has been collected. For example, in a case where there has been an outbreak of an unknown infection, it is also possible to specify information about the place where that infection is happening, and it is possible to change behavior after that in accordance with whether or not there is something being performed at that location.

If data has been collected and a database made, it is next determined whether or not a physician has designated a specified disease (A) (S33). If a physician gives a patient a consultation and diagnoses specified disease (A), a data file DF2 in which that fact is stored is transmitted from the diagnosis and examination institution 9 to the control section 1. In this step, the control section 1 performs determination based on information stored in the datafile DF2. Every time the physician provides diagnosis and consultation, a datafile is transmitted to the control section 1, and may be made into training data. However, this is not limiting and the physician may also designate a disease that they think they are dealing with. As this designated disease, there are chronic cases where there are effects of lifestyle habits etc. and that constitute useful information for other potential patients, cases where there are hereditary factors, or a specified constitution or medical history, that constitute useful information for potential patients of a specified disease having a similar background, or cases of infectiousness that affect a lot of people, and it is necessary to exercise urgency.

If the result of determination in step S33 is that it has been determined that the physician has designated specified disease (A), A patients are searched for from within the DB (S51). Here, the control section 1 (retrieval section 1f) searches for patients who have contracted illness A from within the DB section 8 (refer to FIG. 3).

Next, it is determined whether or not patients of illness A own a lifestyle habit device (S53). As was described previously, individual ID, illness name, and owned devices (devices that can be used) are stored in the DB section 8 (refer to FIG. 3). Here, it is determined whether or not the control section 1 is a device that is being owned (used) by a patient who has contracted illness A.

If the result of determination in step S53 is that such a device is owned, A patients and owned devices are displayed (S55). Here, the control section 1 displays results that have been retrieved from the DB section 8, namely types of devices that are owned by patients having illness A, on the display section 9f of the physician terminal 9e, by means of the diagnosis and examination institution 9 (refer, for example, to FIG. 4A).

If display of owned devices has been performed in step S55, or if the result of determination in step S53 was that no devices were owned, it is next determined whether or not a physician has selected device a (S57). In step S55, if the physician selects a device when the devices have been displayed, a selection result is transmitted to the control section 1 by means of the diagnosis and examination institution 9. Here, it is determined whether or not the physician has selected device based on information from the physician terminal 9e. Since a physician selects a device that is capable of acquiring data that has a meaningful association, in accordance with determination based on their experience and knowledge, in this flow result of the physician's selection is determined. However, this is not limiting and it is also possible to select devices by automating this process, and using all available information, or specific logic, or programs.

If the result of determination in step S57 is that the physician has selected device a, then next, previous information of the selected device a is acquired, and display confirmation may be performed on the physician terminal (S59). Here, the control section 1 acquires previous examination data from the device a that is stored in the DB section 8. This examination data that has been acquired is transmitted to the physician terminal 9e, and may be displayed on the display section 9f. In this case, the physician can confirm examination data of other people that has been acquired using the device a.

Then, training data is made, learning is requested, and an inference model is acquired (S61). Here, the control section 1 makes examination data that was acquired in step S59, and acquired by the device a, into training data (for example, refer to FIG. 5A and FIG. 5B), and issues a request to the learning section 5 by means of the learning request section 6, so as to generate an inference model using this training data. Inference using the inference model here involves input of specified specimen information and bio-information, and acquisition of various diagnosis assisting information as inference results. Also, if the learning section 5 generates an inference model, the inference model is acquired by means of the learning request section 6.

If an inference model has been acquired in step S61, or if the result of determination in step S57 was that the physician did not select device a, it is next determined whether or not there is search completion (S63). Here, the control section 1 determines whether or not the physician has completed the search of step S51. If the result of this determination is that search has not been completed, processing returns to step S53. On the other hand, if search has been completed processing returns to step S31.

Returning to step S33, if the result of determination in this step is that it has been determined that the physician has not designated specified disease (A), it is next determined whether or not there is corresponding data for input to the inference model (S35). Here, the control section 1 determines whether or not there is corresponding data, which is necessary to perform inference using the inference model. For example, determination may be based on whether or not a request for inference has been received from the physician terminal 9e together with examination data. If the result of this determination is that there is no corresponding data, processing returns to step S31.

Also, in a case where there is data related to lifestyle habits, and hereditary and infectious diseases, then since the same disease is likely to happen in a family or the like, data of a family of subjects is input as data for inference, and health advice may be given not for individuals but for that household. Also, in a case where some abnormality has been found at the time of health diagnosis, inference may be performed centered on items related to that abnormality. As a result, data may be collected by designating devices suitable for that illness as monitoring sensors, from among sensors that are being used by those subjects. That is, the control section 1 has a determination section (determination by searching a DB or the like) that determines specified diagnosis results that were subjected to specified diagnosis, among medical examination results of specified people, a symptoms extraction section that extracts symptoms that are dependent on heredity and lifestyle habits, and a determination section that determines monitoring sensors corresponding to symptoms that have been extracted, and collects necessary data by controlling the communication control section 1a so as to perform communication with each device, in accordance with that determination.

Also, in a case where a patient is suffering from cancer or a chronic disease, accuracy at the time of consultation and diagnosis, and at the time of inference, may be improved by using genetic information and microbiome information (one kind of normal bacterial flora) for every patient stored in the DB section 9a (may also be another DB section, or memory storage section of a terminal). For example, these items of information may be stored together with how many types there are, and by simplifying into specified genes and presence or absence information for normal bacterial flora.

On the other hand, if the result of determination in step S35 is that there is corresponding data, inference is performed (S37). As was described previously, inference using the inference model involves input of specified specimen information and bio-information, and acquisition of various diagnosis assisting information as inference results. Here, the control section 1 inputs corresponding data to the input layer of the inference engine 7, and obtains inference results. As inference results, for example, probability or the like of whether a subject is suffering from any kind of illness is output.

If inference has been performed, it is next determined whether or not any results are close to disease A (S39). Here, the control section 1 determines whether or not results are close to illness A based on inference results in step S37. If the result of this determination is not close to illness A, processing returns to step S31.

On the other hand, if the result of determination in step S39 is that there is a result close to illness A, then information is also output to the physician, as required (S41). Here, the control section 1 outputs inference results to the physician terminal 9e by means of the diagnosis and examination institution 9.

Inference results information is also output to determined individuals (S43). In this case, the control section 1 outputs the fact that there are results close to disease A to the terminal 4 owned by the patient. As advice in the case of results that are close to disease A, in a case where prediction inference is possible by analyzing time series biological data etc., there is display to encourage early detailed examination and commencement of treatment.

Also, the DB section 8 collects administration information such as for examination devices and examination kits owned by the medical institutions and examination institutions such as each clinic and hospital etc., making centralized control possible. If it is known what devices exist at what locations, patients and physicians etc. can act to make determination based on accurate information, making it possible to handle problems of excessive infection risk and erroneous diagnosis. If patients and physicians etc. perform advice based on this type of facilities management, it is possible to access a storage section (DB) that stores owned equipment information for every examination and medical institution. The information provision section 1c can transmit effective information in which this kind of holding information and facility information is added, to eligible people. Specifically, in addition to examination data and profile information of the subject people, information dissemination according to owned equipment information for every examination and medical institution becomes possible.

Besides that, it is also possible to perform information output of advice relating to lifestyle habits such as diet, sleep times, exercise etc. “information output of inference results” does not need to be created with inference of all output information, and general information that can be retrieved from inference results may be presented. If information has been output in step S43, or of the result of determination in step S39 is that there are no results close to disease A, processing returns to step S31.

In this way, as shown in FIG. 10A and FIG. 10B, depending on control operation of the control section 1, a physician inputs information relating to symptoms of a patient (refer to S33), a device that is capable of acquiring time series data of the patient is specified (refer to S53 and S55), and time series data and consultation information of another person having a similar device to the specified device is made into training data and a learning request is issued (S59 and S61). As a result, if new symptoms have occurred it is possible to generate an inference model using examination data of other people that use a similar device, and consultation information of those other people. If this inference model is used, then even if new symptoms occur it is possible to use this information for other people as reference information when performing precise consultation.

It should be noted that the processes of steps S59 to S61 are performed in association with the physician, but it is also possible for a computer to perform specified programs in a routine-based manner. Also, with this flow, description has been given on the assumption of artificial intelligence (AI), but it is not absolutely necessary to have inference that uses an inference model from deep learning. There may also be branches and table references using programs, in accordance with specified logic and rules.

As has been described above, in one embodiment and a modified example of the present invention, an input step of a physician inputting symptoms of a specified patient (refer, for example to FIG. 8, S5 in FIG. 9A, and S31 in FIG. 10A), a device specification step of specifying a device that is capable of acquiring previous time series data of a patient (refer, for example, to FIG. 4A, FIG. 4B, and steps S11 and S13 in FIG. 9A), and a learning request step of making time series data of another person having a similar device to the device that has been specified, and consultation information, into training data, and requesting learning (refer, for example, to FIG. 5A, FIG. 5B, FIG. 7A, FIG. 7B, S21 in FIG. 9B, and S61 in FIG. 10B), are executed. As a result, when a physician or the like is giving a consultation, it is possible to simply confirm previous data of a patient, and further, it is possible to easily generate an inference model for illness inference using data of another person who is using a similar device to a device being used by a patient in an examination. Specifically, with this embodiment, when a new event has occurred, it is possible to easily collect information indicating a process leading to this event, and it is possible to create training data for generating an inference model based on this information that has been collected.

Also, with the one embodiment and modified example of the present invention, it becomes possible to propose a device and a method for information transmission, having an examination data acquisition section that acquires examination data of a subject, and a transmitted information determination section that determines transmission information that will be transmitted to the subject, in accordance with owned equipment information for every examination and medical institution, based on profile information of the subject, and information in a storage section that stores the owned equipment information for every examination and medical institution. In this case, owned equipment information may be stored in a DB section 9a of a diagnosis and examination institution, and the owned equipment information may also be stored in the DB section 8.

Also, with the one embodiment and modified example of the present invention, it is possible to provide a device and a method for information transmission that is provided with a first examination data acquisition section that acquires a time series first examination data group of a subject using a first device, and a second examination data acquisition section that acquires a time series second examination data group of the subject using a second device that is capable of examination so as to be able to interpolate the first examination data group, or, by using the first examination data group and the second examination data group, determines transmission information to be presented to the subject. It should be noted that the first examination data group and the second examination data group are not restricted to examination times, or by examination items being mutually supplemented, and abundant analysis and inference, etc. become possible. As was described previously, in a case where time series examination data of a user has been collected using a plurality of devices, then since there are errors and differences in characteristics etc. between individual devices, it is not possible to plot a plurality of sets of time series examination data on the same graph. However, since there are time series examination data for the same subject, inclinations of data change patterns are the same. Therefore, by performing processing to compensate first and examination data that will be subjected to correction computation etc., for a plurality of time series examination data sets, transmission information may be determined using a plurality of time series examination data.

Also, with the one embodiment and modified example of the present invention, it becomes possible to provide an inference system, device, and method, whereby, in an inference system that performs inference using an inference model that has been generated as a result of learning with data that has been collected from many devices as training data, a handling determination section is provided that, in a case where collected data that is other than expected data has been obtained, designates other than expected data, and collects this data that has been designated, and in this way a learning request is issued for an inference model corresponding to the inference system, with at least some of the other than expected data that has been collected being used as training data.

Also, with the one embodiment and modified example of the present invention, it is also possible to provide a sensor determination device and method, whereby, in a case where medical examination results of a specified person are a specified diagnosis, these results are made specified diagnosis results, and symptoms that are dependent on heredity and lifestyle habits are extracted, and monitoring sensors corresponding to the symptoms that have been extracted are determined. In this case, devices that are capable of being utilized by individuals, and functions of those devices, and corresponding cases, are stored in a list of devices that are capable of being used by each of the IDs that are stored in a DB section 8a. Devices that are capable of being used by individuals may be automatically transmitted from a first device 2a, second device 2b, third device 3, diagnosis and examination institution 9, user information section etc., and data that has been input by a user using a questionnaire etc. may be acquired. Also, history data that is stored in the DB section 8a is created for every individual ID for identification of users individually. History data stores dependent relationships, blood relative relationships, medical information, device ID, and acquired data for every ID. symptoms related to heredity and lifestyle habits are often associated with dependent relationships and blood relative relationships, and so monitoring sensors may also be determined for those people.

It should be noted that with the one embodiment and modified example of the present invention, the control section 1, control section 9b, and control section 9h have been described as IT devices comprising a CPU, memory, and HDD etc. However, besides being constructed in the form of software using a CPU and programs, some or all of these sections may be constructed with hardware circuits, or may have a hardware structure such as gate circuitry generated based on a programming language described using Verilog, or may use a hardware structure that uses software, such as a DSP (digital signal processor). Suitable combinations of these approaches may also be used.

Also, without limiting to a CPU, the control section 1, control section 9b, and control section 9h may be components that fulfill functions as a controller, and processing for each of the above described sections may be performed by one or more processors constructed as hardware. For example, each section may be a processor constructed as respective electronic circuits, and may be respective circuits sections of a processor that is constructed with an integrated circuit such as an FPGA (Field Programmable Gate Array). Alternatively, a processor that is constructed with one or more CPUs may execute functions of each section, by reading out and executing computer programs that have been stored in a storage medium.

Also, among the technology that has been described in this specification, with respect to control that has been described mainly using flowcharts, there are many instances where setting is possible using programs, and such programs may be held in a storage medium or storage section. The manner of storing the programs in the storage medium or storage section may be to store at the time of manufacture, or by using a distributed storage medium, or they be downloaded via the Internet.

Also, with the one embodiment of the present invention, operation of this embodiment was described using flowcharts, but procedures and order may be changed, some steps may be omitted, steps may be added, and further the specific processing content within each step may be altered. It is also possible to suitably combine structural elements from different embodiments.

Also, regarding the operation flow in the patent claims, the specification and the drawings, for the sake of convenience description has been given using words representing sequence, such as “first” and “next”, but at places where it is not particularly described, this does not mean that implementation must be in this order.

As understood by those having ordinary skill in the art, as used in this application, ‘section,’ ‘unit,’ ‘component,’ ‘element,’ ‘module,’ ‘device,’ ‘member,’ ‘mechanism,’ ‘apparatus,’ ‘machine,’ or ‘system’ may be implemented as circuitry, such as integrated circuits, application specific circuits (“ASICs”), field programmable logic arrays (“FPLAs”), etc., and/or software implemented on a processor, such as a microprocessor.

The present invention is not limited to these embodiments, and structural elements may be modified in actual implementation within the scope of the gist of the embodiments. It is also possible form various inventions by suitably combining the plurality structural elements disclosed in the above described embodiments. For example, it is possible to omit some of the structural elements shown in the embodiments. It is also possible to suitably combine structural elements from different embodiments.

What is claimed is:

1. A training data collection request device, comprising:
a communication circuit receives information relating to symptoms of a specified patient; and
a processor specifies a device that is capable of acquiring previous time series data of the patient, wherein
the processor acquires, for another person who is using a similar type of device to the device that was specified, data and consultation information that has been collected using the similar type of device to create the training data.

2. The training data collection request device of claim 1, wherein:
the processor makes data that has been collected, and consultation information, into the training data.

3. The training data collection request device of claim 2, wherein:
the processor requests learning for generating an inference model with the training data.

4. The training data collection request device of claim 2, wherein:
the processor creates the training data such that data that has been collected using the similar type of device is input, and the consultation information corresponding to symptoms of the patient is output.

5. The training data collection request device of claim 1, wherein:
the processor specifies the device that is capable of acquiring the previous time series data of the patient, from among devices that have been displayed on a display that displays information, in order to perform selection of the device for data collection.

6. The training data collection request device of claim 3, wherein:
the processor inputs time series data of specified specimen information and/or bio-information to a input of the inference model that has been generated using training data, and the inference model outputs diagnosis assisting information.

7. The training data collection request device of claim 1, wherein:
the processor makes a display indicate devices that are capable of acquiring a plurality of objects, and chronological change in specified information at a plurality of time points, in a list on the display.

8. The training data collection request device of claim 1, wherein:
the processor makes a display indicate chronological change in specified information at a plurality of time points, in a list on the display.

9. The training data collection request device of claim 3, wherein:
the processor makes a display indicate diagnosis assisting information, that has been acquired by inputting previous time series data of a patient to an inference model that was generated.

10. A training data collection device, comprising:
a communication circuit receives information relating to symptoms of a specified patient based on results of having performed diagnosis on the patient; and
a processor specifies a device that is capable of acquiring previous time series data of the patient, wherein
the processor acquires, for another person who is using a similar type of device to the device that was specified, data and consultation information that has been collected using the similar type of device to create the training data.

11. The training data collection device of claim 10, wherein:
the processor makes data that has been collected, and consultation information, into the training data.

12. The training data collection device of claim 11, wherein:
the processor requests learning for generating an inference model with the training data.

13. A training data collection method, comprising:
receiving information relating to symptoms of a specified patient;
specifying a device that is capable of acquiring previous time series data of the patient; and
requesting collection of time series data of another person having a similar type of device to the device that has been specified to create the training data.

14. A non-transitory computer-readable medium storing a processor executable code, which when executed by at least one processor which is provided in a training data collection device, performs a method, the method comprising:
receiving information relating to symptoms of a specified patient;
specifying a device that is capable of acquiring previous time series data of the patient; and
requesting collection of time series data of another person having a similar type of device to the device that has been specified to create the training data.

15. A non-transitory computer-readable medium storing a processor executable code, which when executed by at least one processor which is provided in a training data collection device, performs a method, the method comprising:
receiving information relating to symptoms of a specified patient based on results of having performed diagnosis on the patient;
specifying a device that is capable of acquiring previous time series data of the patient; and
acquiring, for another person who is using a similar type of device to the device that is specified, data and consultation information that has been collected using the similar type of device to create the training data.
